(19) United States
(12) Patent Application Publication
(10) Pub. No.: US 2024/0384231 A1
(43) Pub. Date: Nov. 21, 2024

(54) MATERIALS AND METHODS FOR ENHANCED STEM-CELL LIKE MEMORY T CELL ENGINEERING

(71) Applicant: Janssen Biotech, Inc., Horsham, PA (US)

(72) Inventors: Madhusudhanan SUKUMAR, Spring House, PA (US); Rajkumar GANESAN, Blue Bell, PA (US); Sanjaya SINGH, Blue Bell, PA (US); Tamas ORAVECZ, Spring House, PA (US)

(21) Appl. No.: 18/554,240

(22) PCT Filed: Apr. 7, 2022
(86) PCT No.: PCT/US2022/023883

§ 371 Date: Oct. 6, 2023

Related U.S. Application Data
(60) Provisional application No. 63/172,595, filed on Apr. 8, 2021.
(60) Provisional application No. 63/172,601, filed on Apr. 8, 2021.
(60) Provisional application No. 63/172,605, filed on Apr. 8, 2021.
(60) Provisional application No. 63/172,610, filed on Apr. 8, 2021.

(51) Int. Cl. C12N 5/0783; A61K 39/00; C07K 16/28

(52) U.S. Cl. CPC C12N 5/0636; A61K 39/4611; A61K 39/4631; A61K 39/464417; C07K 16/2878; C07K 2317/622; C12N 2501/2307; C12N 2501/2315; C12N 2501/2321; C12N 2510/00

(57) ABSTRACT

The present disclosure provides methods for generating stem-cell like memory T (TSCM) cells. The present disclosure also provides cells, pharmaceutical compositions, and their uses in adoptive immunotherapy for treatment of a disease, such as cancer.

### CROSS-REFERENCE TO RELATED APPLICATIONS

This application claims priority to U.S. Provisional Application Nos. 63/172,595, filed Apr. 8, 2021, 63/172,601, filed Apr. 8, 2021, 63/172,605, filed Apr. 8, 2021, and 63/172,610, filed Apr. 8, 2021, the disclosure of each of which is herein incorporated by reference in its entirety.

### FIELD OF THE INVENTION

The present invention relates to methods of generating stem-cell like memory T (T_{SCM}) cells for use in adoptive immunotherapy. The present invention also relates to cells, pharmaceutical compositions, and their uses in adoptive immunotherapy for treatment of a disease.

### SEQUENCE LISTING

The instant application contains a Sequence Listing which has been submitted electronically in ASCII format and is hereby incorporated by reference in its entirety. Said ASCII copy, created on Apr. 6, 2022, is named 253505_000131_SL.txt and is 21,082 bytes in size.

### BACKGROUND

Immunotherapy offers a new way to treat solid tumor and other cancers^{1,2}. Biologics including monoclonal antibodies, T-cell redirection bispecific antibodies, checkpoint blockade and, more recently, chimeric antigen receptor-T cells (CAR-T cells), have greatly improved treatment of tumors. Strong evidence suggests that immunotherapy using adoptive transfer of genetically modified T cells such as T-cell receptor (TCR)-transduced and CAR-engineered T cells, can result in complete regression in some patients with metastatic cancer. Currently, four CAR-T therapies have been approved by the Food and Drug Administration (FDA), with more in the clinical pipeline³. However, recent successes with CAR-T cell-based therapies are not without their drawbacks⁴⁻⁷.

### SUMMARY OF THE INVENTION

CAR-T cells are generated by collecting patient's blood, extracting T cells, and expressing CAR, commonly with single-chain fragment variables (scFv) that targets tumor associated antigen(s) (TAA). This process reprograms the patient's T cells to specifically target tumor cells and destroy them, leading to cell death⁸. Current clinical trials have utilized peripheral blood mononuclear cell (PBMC)-derived T cell subsets or unselected bulk T cell populations as a starting population for TCR-engineered and CAR-T cell expansion. Yet current manufacturing processes have led to the generation of inconsistent, variable cell compositions of the final cell product that is administered to the patient.

Stem-cell like memory T (T_{SCM}) cells are a rare population of early memory T cell subset generated directly from naïve T cells, with a distinct phenotypic, transcriptional, and epigenetic state versus other characterized memory and effector T cell subsets. A single T_{SCM} cell possesses the ability to self-renew, thereby reconstituting entire T cell subsets including central memory (T_{CM}), effector memory, and effector T cell subsets. T_{SCM} cells are detected both in healthy donors and cancer patients, albeit at low frequency in the latter, and display gene signatures with fewer exhaustion markers as compared to other known memory T cell subsets. Efforts to generate optimized T_{SCM} or T_{SCM}-like cells from bulk unsorted PBMC populations, in particular applying present-day conventional manufacturing approaches, has proven largely ineffective.

Against this backdrop, the present application provides a method to generate T_{SCM} CAR-T cells with enhanced effector function and decreased exhaustion markers to enhance anti-tumor immunity.

In one aspect, provided herein is a method of enriching stem-cell like memory T (T_{SCM}) cells in a population of T cells, comprising the following step(s):
a) contacting the population of T cells with an effective amount of one or more cytokines comprising Interleukin 7 (IL-7) for a period of time sufficient to enrich T_{SCM} cells; and
b) optionally expanding said T_{SCM} cells.

In some embodiments, the one or more cytokines further comprise IL-15. In some embodiments, the one or more cytokines further comprise IL-21. In some embodiments, the one or more cytokines further comprise IL-15 and IL-21.

In some embodiments, each of the one or more cytokines is contacted with the population of T cells at a concentration of about 1 to 15 ng/ml, about 2 to 14 ng/ml, about 3 to 13 ng/ml, about 4 to 12 ng/ml, about 5 to 12 ng/ml, about 6 to 12 ng/ml, about 7 to 11 ng/ml, about 8 to 12 ng/ml, about 8 to 10 ng/ml, or about 10 ng/ml. In one embodiment, each of the one or more cytokines is contacted with the population of T cells at a concentration of about 10 ng/ml.

In some embodiments, the one or more cytokines do not comprise IL-2.

In some embodiments, the population of T cells comprises Pan T cells, naïve CD4⁺ T cells, naïve CD8⁺ T cells, or naïve CD4⁺ and naïve CD8⁺ T cells, or any combination thereof. In some embodiments, the method further comprises isolating Pan T cells, naïve CD4⁺ cells, naïve CD8⁺ T cells, or naïve CD4⁺ and naïve CD8⁺ cells, or any combination thereof, from peripheral blood monocyte cells (PBMC) prior to step (a). In some embodiments, the population of T cells do not comprise inhibitory, regulatory T cells.

In some embodiments, the one or more cytokines are present during the expansion step (b).

In some embodiments, the method further comprises: genetically modifying the T cells to express a chimeric antigen receptor (CAR) or engineered T cell receptor (TCR). In some embodiments, the genetic modification is conducted by introducing into the cells a polynucleotide encoding said CAR or engineered TCR. In some embodiments, the polynucleotide encoding said CAR or engineered TCR is introduced via viral transduction, electroporation, direct injection, magnetofection, ultrasound, a ballistic or hydrodynamic method, or a combination thereof.

In some embodiments, the CAR or engineered TCR specifically binds a tumor antigen, an infectious antigen or an autoimmune antigen. In some embodiments, the tumor antigen is selected from BCMA, GPRC5D, CD79, KLK2, CD19, CD30, CD33, CD123, hK2, FLT3, CD20, CD22, KRASG12D, p53, BRAC1 and PSMA.

In some embodiments, the genetic modification is conducted prior to the expansion step (b). In some embodiments, the one or more cytokines are present during the genetic modification step.

In various embodiments, the contacting step (a) is performed for about 5-20 days, about 10-20, about 5-18 days, about 8-15 days, about 10-18 days, about 11 days, about 12 days, about 13 days, about 14 days, about 15 days, about 16 days, about 17 days, or about 18 days. In one embodiment, the contacting step (a) is performed for about 14 days.

In various embodiments, the contacting step (a) and the expansion step (b) are performed for a total of 5-20 days, about 10-20, about 5-18 days, about 8-15 days, about 10-18 days, about 11 days, about 12 days, about 13 days, about 14 days, about 15 days, about 16 days, about 17 days, or about 18 days. In some embodiments, the contacting step (a) and the expansion step (b) are performed for a total of about 14 days.

In various embodiments, the contacting step (a) is performed at a temperature of about 37° C.

In various embodiments, the method further comprises: activating the population of T cells at the beginning of the contacting step (a). In one embodiment, the activating step is performed with an anti-CD3 agent and/or an anti-CD28 agent for about 24 hours.

In various embodiments, the method further comprises: priming the population of T cells prior to the activating step (a).

In various embodiments, the method further comprises: determining the percentage of T_{SCM} cells in the population of T cells after the expansion step (b). In some embodiments, the percentage of T_{SCM} cells in the population of T cells is at least about 40%, 50%, 60%, or 70% after the expansion step (b). In one embodiment, the percentage of T_{SCM} cells in the population of T cells is about 60%-70% after the expansion step (b).

In some embodiments, the method for enriching T_{SCM} cells in a population of T cells is performed in vitro or ex vivo.

In some embodiments, provided herein is a method of generating genetically modified stem-cell like memory T (T_{SCM}) cells, comprising the following steps:
a) obtaining a population of isolated Pan T cells, naïve CD4⁺ T cells, naïve CD8⁺ T cells, or naïve CD4⁺ and naïve CD8⁺ T cells, or any combination thereof;
b) activating said population of T cells;
c) genetically modifying the cells present after step (b) to express a chimeric antigen receptor (CAR) or engineered T cell receptor (TCR);
d) expanding said genetically modified cells;
wherein steps b), c) and d) are performed in the presence of one or more cytokines comprising Interleukin-7 (IL-7).

In some embodiments, the one or more cytokines further comprise IL-15. In some embodiments, the one or more cytokines further comprise IL-21. In some embodiments, the one or more cytokines further comprise IL-15 and IL-21.

In some embodiments, each of the one or more cytokines is contacted with the population of T cells at a concentration of about 1 to 15 ng/ml, about 2 to 14 ng/ml, about 3 to 13 ng/ml, about 4 to 12 ng/ml, about 5 to 12 ng/ml, about 6 to 12 ng/ml, about 7 to 11 ng/ml, about 8 to 12 ng/ml, about 8 to 10 ng/ml, or about 10 ng/ml. In one embodiment, each of the one or more cytokines is contacted with the population of T cells at a concentration of about 10 ng/ml.

In various embodiments, the one or more cytokines do not comprise IL-2.

In various embodiments, the Pan T cells, naïve CD4⁺ T cells, naïve CD8⁺ T cells, or naïve CD4⁺ and naïve CD8⁺ T cells, or any combination thereof, are isolated from peripheral blood monocyte cells (PBMC). In some embodiments, the T cells do not comprise inhibitory, regulatory T cells.

In various embodiments, the genetic modification is conducted by introducing into the cells a polynucleotide encoding said CAR or engineered TCR. In some embodiments, the polynucleotide encoding said CAR or engineered TCR is introduced via viral transduction, electroporation, direct injection, magnetofection, ultrasound, a ballistic or hydrodynamic method, or a combination thereof. In some embodiments, the CAR or engineered TCR specifically binds a tumor antigen, an infectious antigen or an autoimmune antigen. In some embodiments, the tumor antigen is selected from BCMA, GPRC5D, CD79, KLK2, CD19, CD30, CD33, CD123, hK2, FLT3, CD20, CD22, KRASG12D, p53, BRAC1 and PSMA.

In some embodiments, the expansion step (d) is performed for 5-20 days, about 10-20, about 5-18 days, about 8-15 days, about 10-18 days, about 11 days, about 12 days, about 13 days, about 14 days, about 15 days, about 16 days, about 17 days, or about 18 days. In some embodiments, the expansion step (d) is performed for about 14 days.

In some embodiments, the steps (b), (c) and (d) are performed for a total of 5-20 days, about 10-20, about 5-18 days, about 8-15 days, about 10-18 days, about 11 days, about 12 days, about 13 days, about 14 days, about 15 days, about 16 days, about 17 days, or about 18 days. In some embodiments, the steps (b), (c) and (d) are performed for a total of 14 days.

In various embodiments, the steps (b), (c) and (d) are performed at a temperature of about 37° C.

In various embodiments, the activating step is performed with an anti-CD3 agent and/or an anti-CD28 agent. In various embodiments, the activating step is performed for about 12-48 hours (e.g., 24 hours).

In various embodiments, the method further comprises: priming the population of T cells prior to the activating step (b).

In various embodiments, the method further comprises: determining the percentage of T_{SCM} cells in the population of T cells after the expansion step (d). In some embodiments, the percentage of T_{SCM} cells in the population of T cells is at least about 40%, 50%, 60%, or 70% after the expansion step (d). In one embodiment, the percentage of T_{SCM} cells in the population of T cells is about 60%-70% after the expansion step (d).

In some embodiments, the method for generating genetically modified T_{SCM} cells is performed in vitro or ex vivo.

In another aspect, provided herein is a population of T cells comprising enriched stem-cell like memory T (T_{SCM}) cells, prepared by a method comprising the following step(s):
a) contacting Pan T cells, naïve CD4⁺ T cells, naïve CD8⁺ T cells, or naïve CD4⁺ and naïve CD8⁺ T cells, or any combination thereof, with an effective amount of one or more cytokines comprising Interleukin 7 (IL-7) for a period of time sufficient to enrich T_{SCM} cells; and
b) optionally expanding said T_{SCM} cells.

In another aspect, provided herein is a population of T cells comprising enriched stem-cell like memory T (T_{SCM}) cells, obtainable by a method comprising the following step(s):
a) contacting Pan T cells, naïve CD4⁺ T cells, naïve CD8⁺ T cells, or naïve CD4⁺ and naïve CD8⁺ T cells, or any combination thereof, with an effective amount of one or more cytokines comprising Interleukin 7 (IL-7) for a period of time sufficient to enrich T_{SCM} cells; and
b) optionally expanding said T_{SCM} cells.

In some embodiments of the population of T cells described herein, the one or more cytokines further comprise IL-15. In some embodiments, the one or more cytokines further comprise IL-21. In some embodiments, the one or more cytokines further comprise IL-15 and IL-21.

In some embodiments of the population of T cells described herein, the one or more cytokines are each added at a concentration of about 1 to 15 ng/ml, about 2 to 14 ng/ml, about 3 to 13 ng/ml, about 4 to 12 ng/ml, about 5 to 12 ng/ml, about 6 to 12 ng/ml, about 7 to 11 ng/ml, about 8 to 12 ng/ml, about 8 to 10 ng/ml, or about 10 ng/ml. In one embodiment, the one or more cytokines are each added at a concentration of about 10 ng/ml.

In some embodiments of the population of T cells described herein, the one or more cytokines do not comprise IL-2.

In some embodiments of the population of T cells described herein, the Pan T cells, naïve CD4⁺ cells, naïve CD8⁺ T cells, or naïve CD4⁺ and naïve CD8⁺ cells, or any combination thereof, are isolated from peripheral blood monocyte cells (PBMC). In some embodiments, the Pan T cells, naïve CD4⁺ cells, naïve CD8⁺ T cells, or naïve CD4⁺ and naïve CD8⁺ cells, or any combination thereof, do not comprise inhibitory, regulatory T cells.

In some embodiments of the population of T cells described herein, the one or more cytokines are present during the expansion step (b).

In some embodiments of the population of T cells described herein, the preparation method further comprises: genetically modifying the T cells to express a chimeric antigen receptor (CAR) or engineered T cell receptor (TCR). In some embodiments, the genetic modification is conducted by introducing into the cells a polynucleotide encoding said CAR or engineered TCR. In some embodiments, the polynucleotide encoding said CAR or engineered TCR is introduced via viral transduction, electroporation, direct injection, magnetofection, ultrasound, a ballistic or hydrodynamic method, or a combination thereof.

In some embodiments of the population of T cells described herein, the CAR or engineered TCR specifically binds a tumor antigen, an infectious antigen or an autoimmune antigen.

In some embodiments of the population of T cells described herein, the tumor antigen is selected from BCMA, GPRC5D, CD79, KLK2, CD19, CD30, CD33, CD123, hK2, FLT3, CD20, CD22, KRASG12D, p53, BRAC1 and PSMA.

In some embodiments of the population of T cells described herein, the genetic modification is conducted prior to the expansion step (b). In some embodiments, the one or more cytokines are present during the genetic modification step. In some embodiments, the one or more cytokines are present during the expansion step (b).

In some embodiments of the population of T cells described herein, the contacting step (a) is performed for about 5-20 days, about 10-20, about 5-18 days, about 8-15 days, about 10-18 days, about 11 days, about 12 days, about 13 days, about 14 days, about 15 days, about 16 days, about 17 days, or about 18 days. In one embodiment, the contacting step (a) is performed for about 14 days.

In some embodiments of the population of T cells described herein, the contacting step (a) and the expansion step (b) are performed for a total of about 5-20 days, about 10-20, about 5-18 days, about 8-15 days, about 10-18 days, about 11 days, about 12 days, about 13 days, about 14 days, about 15 days, about 16 days, about 17 days, or about 18 days. In one embodiment, the contacting step (a) and the expansion step (b) are performed for a total of 14 days.

In some embodiments of the population of T cells described herein, the contacting step (a) is performed at a temperature of about 37° C.

In some embodiments of the population of T cells described herein, the preparation method further comprises: activating the population of T cells at the beginning of the contacting step (a). In one embodiment, the activating step is performed with an anti-CD3 agent and/or an anti-CD28 agent for about 24 hours.

In some embodiments of the population of T cells described herein, the method further comprises: priming the population of T cells prior to the activating step (a).

In some embodiments of the population of T cells described herein, the method further comprises: determining the percentage of T_{SCM} cells in the population of T cells after the expansion step (b). In some embodiments, the percentage of T_{SCM} cells in the population of T cells is at least about 40%, 50%, 60%, or 70% after the expansion step (b). In one embodiment, the percentage of T_{SCM} cells in the population of T cells is about 60%-70% after the expansion step (b).

In another aspect, provided herein is a population of T cells comprising enriched stem-cell like memory T (T_{SCM}) cells, prepared by (or obtainable by) a method comprising the following steps:
a) obtaining a population of isolated Pan T cells, naïve CD4⁺ T cells, naïve CD8⁺ T cells, or naïve CD4⁺ and naïve CD8⁺ T cells, or any combination thereof;
b) activating said population of T cells;
c) genetically modifying the cells present after step (b) to express a chimeric antigen receptor (CAR) or engineered T cell receptor (TCR);
d) expanding said genetically modified cells;
wherein steps b), c) and d) are performed in the presence of one or more cytokines comprising Interleukin-7 (IL-7).

In some embodiments of the population of T cells described herein, the step of obtaining a population of isolated Pan T cells, naïve CD4⁺ T cells, naïve CD8⁺ T cells, or naïve CD4⁺ and naïve CD8⁺ T cells, or any combination thereof, is performed on a sample obtained from a subject.

In some embodiments of the population of T cells described herein, the one or more cytokines further comprise IL-15. In some embodiments, the one or more cytokines further comprise IL-21. In some embodiments, the one or more cytokines further comprise IL-15 and IL-21.

In some embodiments of the population of T cells described herein, the one or more cytokines are each added at a concentration of about 1 to 15 ng/ml, about 2 to 14 ng/ml, about 3 to 13 ng/ml, about 4 to 12 ng/ml, about 5 to 12 ng/ml, about 6 to 12 ng/ml, about 7 to 11 ng/ml, about 8 to 12 ng/ml, about 8 to 10 ng/ml, or about 10 ng/ml. In one embodiment, the one or more cytokines are each added at a concentration of about 10 ng/ml.

In various embodiments of the population of T cells described herein, the one or more cytokines do not comprise IL-2.

In various embodiments of the population of T cells described herein, the Pan T cells, naïve CD4⁺ T cells, naïve CD8⁺ T cells, or naïve CD4⁺ and naïve CD8⁺ T cells, or any combination thereof, are isolated from peripheral blood monocyte cells (PBMC). In some embodiments, the T cells do not comprise inhibitory, regulatory T cells.

In various embodiments of the population of T cells described herein, the genetic modification is conducted by introducing into the cells a polynucleotide encoding said CAR or engineered TCR. In some embodiments, the polynucleotide encoding said CAR or engineered TCR is introduced via viral transduction, electroporation, direct injection, magnetofection, ultrasound, a ballistic or hydrodynamic method, or a combination thereof. In some embodiments, the CAR or engineered TCR specifically binds a tumor antigen, an infectious antigen or an autoimmune antigen. In some embodiments, the tumor antigen is selected from BCMA, GPRC5D, CD79, KLK2, CD19, CD30, CD33, CD123, hK2, FLT3, CD20, CD22, KRASG12D, p53, BRAC1 and PSMA.

In various embodiments of the population of T cells described herein, the expansion step (d) is performed for about 5-20 days, about 10-20, about 5-18 days, about 8-15 days, about 10-18 days, about 11 days, about 12 days, about 13 days, about 14 days, about 15 days, about 16 days, about 17 days, or about 18 days. In some embodiments, the expansion step (d) is performed for about 14 days.

In various embodiments of the population of T cells described herein, the steps (b), (c) and (d) are performed for a total of about 5-20 days, about 10-20, about 5-18 days, about 8-15 days, about 10-18 days, about 11 days, about 12 days, about 13 days, about 14 days, about 15 days, about 16 days, about 17 days, or about 18 days. In some embodiments, the steps (b), (c) and (d) are performed for a total of 14 days.

In various embodiments of the population of T cells described herein, the steps (b), (c) and (d) are performed at a temperature of about 37° C.

In various embodiments of the population of T cells described herein, the activating step is performed with an anti-CD3 agent and/or an anti-CD28 agent. In various embodiments, the activating step is performed for about 24 hours.

In various embodiments of the population of T cells described herein, the method further comprises: priming the population of T cells prior to the activating step (b).

In various embodiments of the population of T cells described herein, the method further comprises: determining the percentage of T_{SCM} cells in the population of T cells after the expansion step (d). In some embodiments, the percentage of T_{SCM} cells in the population of T cells is at least about 40%, 50%, 60%, or 70% after the expansion step (d). In some embodiments, the percentage of T_{SCM} cells in the population of T cells is about 60%-70% after the expansion step (d).

In another aspect, provided herein is a pharmaceutical composition comprising the population of T cells described herein, and a pharmaceutically acceptable carrier or excipient.

In another aspect, provided herein is a method of treating a disease or disorder in a subject in need thereof, comprising administering to the subject a therapeutically effective amount of the population of T cells comprising enriched stem-cell like memory T (T_{SCM}) cells, or a pharmaceutical composition comprising said population of T cells comprising enriched T_{SCM} cells and a pharmaceutically acceptable carrier or excipient, wherein said population of T cells comprising enriched T_{SCM} cells are prepared by (or obtainable by) a method comprising the following steps:
a) obtaining a population of isolated Pan T cells, naïve CD4⁺ T cells, naïve CD8⁺ T cells, or naïve CD4⁺ and naïve CD8⁺ T cells, or any combination thereof;
b) activating said population of T cells;
c) genetically modifying the cells present after step (b) to express a chimeric antigen receptor (CAR) or engineered T cell receptor (TCR);
d) expanding said genetically modified cells;
wherein steps b), c) and d) are performed in the presence of one or more cytokines comprising Interleukin-7 (IL-7).

In some embodiments of the treatment method described herein, the step of obtaining a population of isolated Pan T cells, naïve CD4⁺ T cells, naïve CD8⁺ T cells, or naïve CD4⁺ and naïve CD8⁺ T cells, or any combination thereof, is performed on a sample obtained from a subject.

In some embodiments of the treatment method described herein, the one or more cytokines further comprise IL-15. In some embodiments, the one or more cytokines further comprise IL-21. In some embodiments, the one or more cytokines further comprise IL-15 and IL-21.

In some embodiments of the treatment method described herein, the one or more cytokines are each added at a concentration of about 1 to 15 ng/ml, about 2 to 14 ng/ml, about 3 to 13 ng/ml, about 4 to 12 ng/ml, about 5 to 12 ng/ml, about 6 to 12 ng/ml, about 7 to 11 ng/ml, about 8 to 12 ng/ml, about 8 to 10 ng/ml, or about 10 ng/ml. In one embodiment, the one or more cytokines are each added at a concentration of about 10 ng/ml.

In some embodiments of the treatment method described herein, the one or more cytokines do not comprise IL-2.

In some embodiments of the treatment method described herein, the Pan T cells, naïve CD4⁺ T cells, naïve CD8⁺ T cells, or naïve CD4⁺ and naïve CD8⁺ T cells, or any combination thereof, are isolated from peripheral blood monocyte cells (PBMC). In some embodiments, the T cells do not comprise inhibitory, regulatory T cells.

In some embodiments of the treatment method described herein, the genetic modification is conducted by introducing into the cells a polynucleotide encoding said CAR or engineered TCR. In some embodiments, the polynucleotide encoding said CAR or engineered TCR is introduced via viral transduction, electroporation, direct injection, magnetofection, ultrasound, a ballistic or hydrodynamic method, or a combination thereof. In some embodiments, the CAR or engineered TCR specifically binds a tumor antigen, an infectious antigen or an autoimmune antigen. In some embodiments, the tumor antigen is selected from BCMA, GPRC5D, CD79, KLK2, CD19, CD30, CD33, CD123, hK2, FLT3, CD20, CD22, KRASG12D, p53, BRAC1 and PSMA.

In some embodiments of the treatment method described herein, the expansion step (d) is performed for about 5-20 days, about 10-20, about 5-18 days, about 8-15 days, about 10-18 days, about 11 days, about 12 days, about 13 days, about 14 days, about 15 days, about 16 days, about 17 days, or about 18 days. In some embodiments, the expansion step (d) is performed for about 14 days.

In some embodiments of the treatment method described herein, the steps (b), (c) and (d) are performed for a total of about 5-20 days, about 10-20, about 5-18 days, about 8-15 days, about 10-18 days, about 11 days, about 12 days, about 13 days, about 14 days, about 15 days, about 16 days, about 17 days, or about 18 days. In some embodiments, the steps (b), (c) and (d) are performed for a total of 14 days.

In some embodiments of the treatment method described herein, the steps (b), (c) and (d) are performed at a temperature of about 37° C.

In some embodiments of the treatment method described herein, the activating step is performed with an anti-CD3 agent and/or an anti-CD28 agent for about 24 hours.

In some embodiments of the treatment method described herein, the method further comprises: priming the population of T cells prior to the activating step (b).

In some embodiments of the treatment method described herein, the method further comprises: determining the percentage of T_{SCM} cells in the population of T cells after the expansion step (d). In some embodiments, the percentage of T_{SCM} cells in the population of T cells is at least about 40%, 50%, 60%, or 70% after the expansion step (d). In some embodiments, the percentage of T_{SCM} cells in the population of T cells is about 60%-70% after the expansion step (d).

In some embodiments of the treatment method described herein, the population of T cells are allogeneic to the subject. In some embodiments, the population of T cells are autologous to the subject.

In some embodiments of the treatment method described herein, the disease or disorder is a cancer, an infectious disease, or an autoimmune disease. In some embodiments, the cancer is a hematologic malignancy. In some embodiments, the cancer is a solid tumor. In some embodiments, the cancer is squamous cell cancer, adenosquamous cell carcinoma, lung cancer, cancer of the peritoneum, hepatocellular cancer, gastric or stomach cancer, cervical cancer, ovarian cancer, liver cancer, bladder cancer, cancer of the urinary tract, hepatoma, breast cancer, colon cancer, colorectal cancer, endometrial cancer, salivary gland carcinoma, kidney or renal cancer, prostate cancer, vulval cancer, thyroid cancer, hepatic carcinoma, anal carcinoma, penile carcinoma, skin cancer, multiple myeloma and acute lymphocytic leukemia (ALL), acute myelocytic leukemia (AML), chronic myelocytic leukemia (CML), and chronic lymphocytic leukemia (CLL), lymphoma such as Hodgkin lymphoma (HL) and non-Hodgkin lymphoma (NHL), follicular lymphoma, chronic lymphocytic leukemia/small lymphocytic lymphoma (CLL/SLL), mantle cell lymphoma (MCL), marginal zone B-cell lymphomas, primary mediastinal B-cell lymphoma, Burkitt lymphoma, lymphoplasmacytic lymphoma, immunoblastic large cell lymphoma, hairy cell leukemia (HCL), precursor B-lymphoblastic lymphoma and primary central nervous system (CNS) lymphoma, T-cell NHL such as precursor T-lymphoblastic lymphoma/leukemia, peripheral T-cell lymphoma (PTCL), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma, a mixture of one or more leukemia/lymphoma as described above, brain, as well as head and neck cancer, biliary cancer, bronchus cancer, chordoma, choriocarcinoma, epithelial carcinoma, endothelial sarcoma, esophageal cancer, Ewing sarcoma, heavy chain disease, hematopoietic cancer, immunocytic amyloidosis, monoclonal gammopathy of undetermined significance, myelodysplastic syndromes, myeloproliferative disorder, agnogenic myeloid metaplasia (AMM) or myelofibrosis (MF), chronic idiopathic myelofibrosis, myeloproliferative neoplasms, polycythemia vera, rectum adenocarcinoma, essential thrombocytosis, chronic neutrophilic leukemia, hypereosinophilic syndrome, or soft tissue sarcoma, or a combination or metastases thereof.

In some embodiments, the cancer is a BCMA-expressing cancer. In some embodiments, the BCMA-expressing cancer is acute myeloid leukemia (AML) or multiple myeloma (MM), or smoldering multiple myeloma (SMM).

In another aspect, provided herein is a system for enriching stem-cell like memory T (T_{SCM}) cells in a population of T cells, comprising the following elements:
a) a means for contacting the population of T cells with an effective amount of one or more cytokines comprising Interleukin 7 (IL-7) for a period of time sufficient to enrich T_{SCM} cells; and
b) optionally a means for expanding said T_{SCM} cells.

In some embodiments of the system described herein, the population of T cells comprises Pan T cells, naïve CD4⁺ T cells, naïve CD8⁺ T cells, or naïve CD4⁺ and naïve CD8⁺ T cells, or any combination thereof.

In some embodiments of the system described herein, the system further comprises means for isolating Pan T cells, naïve CD4⁺ cells, naïve CD8⁺ T cells, or naïve CD4⁺ and CD8⁺ naïve cells, or any combination thereof, from peripheral blood monocyte cells (PBMC). In some embodiments, the system further comprises means for genetically modifying the T cells to express a chimeric antigen receptor (CAR) or engineered T cell receptor (TCR). In some embodiments, the system further comprises means for activating the population of T cells at the beginning of the contacting said population of T cells with the one or more cytokines comprising Interleukin 7 (IL-7). In some embodiments, the system further comprises means for priming the population of T cells prior to the activating. In some embodiments, the system further comprises means for determining the percentage of T_{SCM} cells in the population of T cells after the expansion.

In another aspect, provided herein is a system for generating genetically modified stem-cell like memory T (T_{SCM}) cells, comprising the following elements:
a) means for obtaining a population of isolated Pan T cells, naïve CD4⁺ T cells, naïve CD8⁺ T cells, or naïve CD4⁺ and naïve CD8⁺ T cells, or any combination thereof,
b) means for activating said population of T cells;
c) means for genetically modifying the cells after activating to express a chimeric antigen receptor (CAR) or engineered T cell receptor (TCR);
d) means for expanding said genetically modified cells;
wherein obtaining a population of isolated Pan T cells, naïve CD4⁺ T cells, naïve CD8⁺ T cells, or naïve CD4⁺ and naïve CD8⁺ T cells, or any combination thereof, activating said population of T cells and genetically modifying said population of T cells is performed in the presence of one or more cytokines comprising Interleukin-7 (IL-7).

In various embodiments of the system described herein, the one or more cytokines further comprise IL-15 and/or IL-21.

In another aspect, provided herein is a composition for enriching stem-cell like memory T (T_{SCM}) cells in a population of T cells, comprising:
a. a population of T cells,
b. one or more cytokines comprising Interleukin 7 (IL-7), which is a means for enriching T_{SCM} cells and
c. optionally a means for expanding said T_{SCM} cells.

In another aspect, provided herein is a composition for enriching stem-cell like memory T (T_{SCM}) cells in a population of T cells, comprising:
a. a population of T cells, and
b. an effective amount of one or more cytokines comprising Interleukin 7 (IL-7), and a means for:
(i) contacting the population of T cells with an effective amount of one or more cytokines comprising IL-7 thereby enriching T_{SCM} cells,
(ii) activating said enriched T_{SCM} cells, and
(iii) optionally a means for expanding said T_{SCM} cells.

In another aspect, provided herein is a composition for generating genetically modified stem-cell like memory T (T_{SCM}) cells, comprising:
a. a population of T cells, and
b. an effective amount of one or more cytokines comprising Interleukin 7 (IL-7), and
a means for:
(i) contacting the population of T cells with an effective amount of one or more cytokines comprising IL-7 thereby enriching T_{SCM} cells,
(ii) activating said enriched T_{SCM} cells,
(iii) genetically modifying said enriched T_{SCM} cells to express a chimeric antigen receptor (CAR) or engineered T cell receptor (TCR), and
(iv) optionally a means for expanding said T_{SCM} cells.

In various embodiments, the T_{SCM} cells are enriched by placing the population of T cells in contact with an effective amount of the one or more cytokines comprising IL-7 for a period of time sufficient to enrich T_{SCM} cells.

### BRIEF DESCRIPTION OF THE DRAWINGS

FIGS. 1A-1B show an exemplary second generation CAR-T cell design. FIG. 1A is diagram of a second-generation CAR-T comprising a scFv targeting a TAA of interest, e.g., B-cell maturation antigen (BCMA), fused with a hinge and transmembrane sequence derived from human CD8A, and 4-1BB (CD137) and CD3(intracellular domains. FIG. 1B shows the amino acid sequences of the exemplary anti-BCMA CAR constructs described herein.

FIGS. 2A-2C show that cytokine conditioning enhances T_{SCM} cell phenotype in CD4⁺ T cell subset. Pan-T cells were activated in the presence or absence of the indicated cytokines. For fluorescence-activated cell sorting (FACS) analysis, the cells were gated on CD4⁺ CAR⁺ T cells. Frequency of the expression levels of CD62L (FIG. 2A), CCR7 (FIG. 2B) and CD27 (FIG. 2C) within the CD4⁺ BCMA-HL CAR-transduced cells was determined. Shown here is the phenotypic characterization at day 14 after CAR-T cell transduction.

FIGS. 3A-3B show that cytokine conditioning enhances T_{SCM} cell phenotype in CD8⁺ T cell subset. Pan-T cells were activated in the presence or absence of the indicated cytokines. For FACS analysis, the cells were gated on CD8⁺ CAR⁺ T cells. Frequency of the expression levels of CD62L (FIG. 3A), CCR7 (FIG. 3B), and CD27 (FIG. 3C) levels within the CD8⁺ BCMA-HL CAR-transduced cells was determined. Shown here is the phenotypic characterization at day 14 after CAR-T cell transduction.

FIGS. 4A-4B show that cytokine conditioning enhances CD45RO⁻/CD45RA⁻ T_{SCM} cell phenotype in both CD4⁺ and CD8⁺ T cell subsets. Pan-T cells were activated in the presence or absence of the indicated cytokines. For FACS analysis, the cells were gated on CAR⁺ T cells. Frequency of the expression levels of CD45RO⁻/CD45RA⁺ in CD4⁺ CAR⁺ T cell subset (FIG. 4A). Frequency of the expression levels of CD45RO⁻/CD45RA⁺ in CD8⁺ CAR⁺ T cell subset (FIG. 4B). Shown here is the phenotypic characterization at day 14 after CAR-T cell transduction.

### DETAILED DESCRIPTION

### Definitions

The terms “T cell” and “T lymphocyte” are interchangeable and used synonymously herein. As used herein, T cell includes thymocytes, naïve T lymphocytes, immature T lymphocytes, mature T lymphocytes, resting T lymphocytes, or activated T lymphocytes. A T cell can be a T helper (Th) cell, for example a T helper 1 (Th1), a T helper 2 (Th2) cell, a T helper 17 (Th17) or regulatory T (Treg) cell. The T cell can be a T helper cell (Th; CD4⁺ T cell) CD4⁺ T cell, CD8⁺ T cell, a cytotoxic T cell (CTL; CD8⁺ T cell), a tumor infiltrating cytotoxic T cell (TIL; CD8⁺ T cell), CD4+CD8⁺ T cell, stem-cell like memory T (T_{SCM}) cells, central memory T cells (T_{CM}), effector memory T cells (T_{EM}), terminal effector T cells (T_{eff}), or any other subset of T cells. Illustrative populations of T cells suitable for use in particular embodiments include stem central memory T cells (T_{SCM}).

Naïve T cells can have the following expression pattern of cell surface markers: CCR7⁺, CD62L⁺, CD45RA⁺, CD45RO⁻, CD95⁺. Stem-cell like memory T cells (T_{SCM}) can have the following expression pattern of cell surface markers: CCR7⁺, CD62L⁺, CD45RA⁺, CD45RO⁻, CD95⁺. Central memory T cells (T_{CM}) can have the following expression pattern of cell surface markers: CCR7⁺, CD62L⁺, CD45RA, CD45RO⁺, CD95⁺. Effector memory T cells (T_{EM}) can have the following expression pattern of cell surface markers: CCR7⁻, CD62L⁻, CD45RA⁻, CD45RO⁺, CD95⁺. Terminal effector T cells (T_{eff}) can have the following expression pattern of cell surface markers: CCR7⁻, CD62L⁻, CD45RO⁻, CD95⁺. See, e.g., Gattinoni et al. Nat. Med. 17(2011):1290-7; and Flynn et al. Clin. Translat. Immunol. 3(2014):e20, which are incorporated herein by reference in their entirety for all purposes.

The terms “express” and “expression” mean allowing or causing the information in a gene or DNA sequence to become produced, for example producing an RNA or a protein by activating the cellular functions involved in transcription and/or translation of a corresponding gene or DNA sequence. A DNA sequence is expressed in or by a cell to form an “expression product” such as an RNA or a protein. The expression product itself, e.g., the resulting protein, may also be said to be “expressed” by the cell. An expression product can be characterized as intracellular, extracellular or transmembrane.

The terms “vector”, “cloning vector” and “expression vector” mean the vehicle by which a DNA or RNA sequence (e.g., a foreign gene) can be introduced into a host cell, so as to genetically modify the cell and promote expression (e.g., transcription and translation) of the introduced sequence. Vectors include plasmids, synthesized RNA and DNA molecules, phages, viruses, etc. In certain embodiments, the vector is a viral vector such as, but not limited to, viral vector is an adenoviral, adeno-associated, alphaviral, herpes, lentiviral, retroviral, or vaccinia vector.

As used herein, the terms “specifically binds”, “specifically recognizes”, or “specific for” refer to measurable and reproducible interactions such as binding between a target and an antigen binding protein (such as a CAR or an engineered TCR), which is determinative of the presence of the target in the presence of a heterogeneous population of molecules including biological molecules.

The term “polypeptide,” “peptide” or “protein” are used interchangeably and to refer to a polymer of amino acid residues. The terms encompass all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation, pegylation, biotinylation, etc.).

The terms “nucleic acid”, “nucleotide”, and “polynucleotide” encompass both DNA and RNA unless specified otherwise. By a “nucleic acid sequence” or “nucleotide sequence” is meant the nucleic acid sequence encoding an amino acid, the term may also refer to the nucleic acid sequence including the portion coding for any amino acids added as an artifact of cloning, including any amino acids coded for by linkers.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition, but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof, or (3) relieving the disease, i.e., causing regression of the state, disorder or condition or at least one of its clinical or sub-clinical symptoms. The benefit to a subject to be treated is either statistically significant or at least perceptible to the patient or to the physician.

The term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The phrase “pharmaceutically acceptable”, as used in connection with compositions described herein, refers to molecular entities and other ingredients of such compositions that are physiologically tolerable and do not typically produce untoward reactions when administered to a mammal (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

The terms “patient”, “individual”, “subject”, and “animal” are used interchangeably herein and refer to mammals, including, without limitation, human, non-human primates, and veterinary animals (e.g., cats, dogs, cows, horses, sheep, pigs, etc.) and experimental animal models. Examples of mammals include mammals of the order Rodentia, such as mice and hamsters, mammals of the order Lagomorpha, such as rabbits, mammals of the order Carnivora, including felines (cats) and canines (dogs), mammals of the order Artiodactyla, including bovines (cows) and swines (pigs), mammals of the order Perissodactyla, including equines (horses), or mammals of the order Primates, Ceboids, or Simoids (monkeys) or of the order Anthropoids (humans and apes). In a preferred embodiment, the subject is a human.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

Singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

Throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

All embodiments of any aspect of the invention can be used in combination, unless the context clearly dictates otherwise.

### Methods of the Invention

Cells suitable for use in the methods of the present disclosure can come from all cells and tissues, and particularly mammalian cells and tissues. Suitable cells may have human, ape, monkey, porcine, or rodent origin and may be primary cells or cultured cells. In some embodiments, the cells that are modified using the methods of the present disclosure are human cells.

### Isolation/Enrichment of Donor Cells

In some embodiments, cells used in the methods of the present disclosure are obtained from a donor. In some embodiments, the cells may be allogeneic or non-autologous (“non-self”) with respect to the recipient to whom the cells are administered. In alternative embodiments, the cells may be autologous with respect to the recipient to whom the cells are administered. In some embodiments, the cells are obtained from a mammalian subject. In other embodiments, the cells are obtained from a primate subject. In some embodiments, the cells are obtained from a human subject.

In some embodiments, the cells used in the methods of the present disclosure are lymphocytes (e.g., T cells). Lymphocytes can be obtained from sources such as, but not limited to, peripheral blood mononuclear cells, bone marrow, lymph nodes tissue, cord blood, thymus issue, tissue from a site of infection, ascites, pleural effusion, spleen tissue, and tumors. Lymphocytes may also be generated by differentiation of stem cells. In some embodiments, lymphocytes can be obtained from blood collected from a subject using techniques generally known to the skilled person, such as sedimentation, e.g., FICOLL™ separation.

Cells from the circulating blood of a subject can be obtained by apheresis. An apheresis device typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media for subsequent processing. The cells can be washed with PBS or with another suitable solution that lacks calcium, magnesium, and most, if not all other, divalent cations. A washing step may be accomplished by methods known to those in the art, such as, but not limited to, using a semiautomated flowthrough centrifuge (e.g., Cobe 2991 cell processor, or the Baxter CytoMate). After washing, the cells may be resuspended in a variety of biocompatible buffers, cell culture medias, or other saline solution with or without buffer.

T cells can be isolated from peripheral blood mononuclear cells (PBMCs) by lysing the red blood cells and depleting the monocytes. As an example, T cells can be sorted by centrifugation through a PERCOLL™ gradient. In some embodiments, after isolation of PBMC, both cytotoxic and helper T lymphocytes can be sorted into naïve, memory, and effector T cell subpopulations either before or after activation, expansion, and/or genetic modification.

In some embodiments, the population of T cells used in the methods described herein comprises Pan T cells, naïve CD4⁺ T cells, naïve CD8⁺ T cells, or naïve CD4⁺ and CD8⁺ T cells, or any combination thereof. In some embodiments, the population of T cells are isolated from a sample obtained from a subject.

In some embodiments, the population of T cells used in the methods described herein does not comprise inhibitory, regulatory T cells.

In some embodiments, T lymphocytes can be enriched. For example, a specific subpopulation of T lymphocytes, such as stem central memory T cells (T_{SCM}), expressing one or more markers such as, but not limited to, CD3, CD4, CD8, CD14, CD15, CD16, CD19, CD27, CD28, CD34, CD36, CD45RA, CD45RO, CD56, CD62, CD62L, CD122, CD123, CD127, CD235a, CCR7, HLA-DR or a combination thereof, can be enriched using either positive or negative selection techniques. In some embodiments, stem-cell like memory T cells (T_{SCM}) are enriched using the methods detailed herein.

In some embodiments, the T lymphocytes can also be differentiated from stem cells, such as cord blood stem cells, progenitor cells, bone marrow stem cells, hematopoietic stem cells (HSCs) and induced pluripotent stem cells (iPSCs).

### Genetic Modifications

The T cells may be genetically modified to express high affinity T cell receptors (engineered TCRs) or chimeric antigen receptors (CARs). In some embodiments, methods described herein include the step of introducing into cells an exogenous nucleic acid molecule comprising a nucleotide sequence coding for a CAR or an engineered TCR. In some embodiments, the T cells are genetically modified to express one or more engineered TCRs or CARs.

In some embodiments, genetically modification may be performed in the presence of one or more cytokines comprising IL-7, IL-15 and/or IL-21. In some embodiments, genetically modification may be performed in the presence of IL-7. In some embodiments, genetically modification may be performed in the presence of IL-7 and IL-15. In some embodiments, genetically modification may be performed in the presence of IL-7 and IL-21. In some embodiments, genetically modification may be performed in the presence of IL-7, IL-15 and IL-21.

In some embodiments, genetically modification may be performed in the absence of IL-2.

In some embodiments, genetically modification may be performed prior to expansion of T cells. For example, the Pan T cells, naïve CD4⁺ T cells, naïve CD8⁺ T cells, or naïve CD4⁺ and CD8⁺ T cells, or any combination thereof isolated from PBMCs may be genetically modified.

In some embodiments, genetically modification may be performed after expansion of T cells. In some embodiments, T cells comprising the enriched stem-cell like memory T cells (T_{SCM}) are genetically modified.

A “chimeric antigen receptors” or “CAR” as used herein refers to a cell-surface receptor comprising an extracellular target-binding domain, a transmembrane domain and a cytoplasmic domain which comprises a lymphocyte activation domain and optionally at least one co-stimulatory signaling domain, all in a combination that is not naturally found together on a single protein. This particularly includes receptors wherein the extracellular domain and the cytoplasmic domain are not naturally found together on a single receptor protein.

Naturally occurring T cell receptors comprise two subunits, an α-subunit and β-subunit, each of which is a unique protein produced by recombination event in each T cell's genome. Libraries of TCRs may be screened for their selectivity to particular target antigens. In this manner, natural TCRs, which have a high-avidity and reactivity toward target antigens may be selected, cloned, and subsequently introduced into a population of T cells used for adoptive immunotherapy.

In one embodiment, T cells are modified by introducing a polynucleotide encoding a subunit of a TCR that has the ability to form TCRs that confer specificity to T cells for tumor cells expressing a target antigen. In particular embodiments, the subunits have one or more amino acid substitutions, deletions, insertions, or modifications compared to the naturally occurring subunit, so long as the subunits retain the ability to form TCRs conferring upon transfected T cells the ability to home to target cells, and participate in immunologically-relevant cytokine signaling. The engineered TCRs preferably also bind target cells displaying the relevant tumor-associated peptide with high avidity, and optionally mediate efficient killing of target cells presenting the relevant peptide in vivo.

The exogenous nucleic acid molecule comprising a nucleotide sequence coding for a CAR or an engineered TCR may be episomally expressed. Alternatively, the exogenous nucleic acid molecule comprising a nucleotide sequence coding for a CAR or an engineered TCR may be knocked into a gene locus via homology directed repair (HDR) (e.g., by using a genome editing nuclease such as CRISPR/Cas). For example, and not by limitation, the exogenous nucleic acid molecule comprising a nucleotide sequence coding for a CAR or an engineered TCR may be knocked into a TCR alpha, TCR beta, or B2M locus to replace the endogenous gene. In the case of knock-in, the nucleic acid molecule comprising a nucleotide sequence coding for a CAR or an engineered TCR may be provided as a double stranded DNA (dsDNA), a single-stranded DNA (ssDNA), or in a viral vector (e.g., AAV). In either embodiments, the gene is operatively linked (i.e., under transcriptional control) to a promoter active in the cells.

The CAR or the engineered TCR may be directed against an antigen expressed at the surface of a malignant cell, or an infected cell, such as a tumor antigen or an infectious antigen.

Non-limiting examples of tumor antigens that may be targeted by the modified cells described herein include B-cell maturation antigen (BCMA), human epidermal growth factor receptor 2 (HER2), Kallikrein Related Peptidase 2 (KLK2), Hexokinase 2 (hK2), interleukin-13 receptor subunit alpha-2 (IL-13Ra2), ephrin type-A receptor 2 (EphA2), A kinase anchor protein 4 (AKAP-4), adrenoceptor beta 3 (ADRB3), anaplastic lymphoma kinase (ALK), immunoglobulin lambda-like polypeptide 1 (IGLL1), androgen receptor, angiopoietin-binding cell surface receptor 2 (Tie 2), B7H3 (CD276), bone marrow stromal cell antigen 2 (BST2), carbonic anhydrase IX (CAIX), CCCTC-binding factor (Zinc Finger Protein)-like (BORIS), CD171, CD179a, CD24, CD300 molecule-like family member f (CD300LF), CD38, CD44v6, CD72, CD79a, CD79b, CD97, chromosome X open reading frame 61 (CXORF61), claudin 6 (CLDN6), CS-1 (CD2 subset 1, CRACC, SLAMF7, CD319, or 19A24), C-type lectin domain family 12 member A (CLEC12A), C-type lectin-like molecule-1 (CLL-1), Cyclin B 1, Cytochrome P450 1B 1 (CYP1B 1), EGF-like module-containing mucin-like hormone receptor-like 2 (EMR2), epidermal growth factor receptor (EGFR), ERG (transmembrane protease, serine 2 (TMPRSS2) ETS fusion gene), ETS translocation-variant gene 6, located on chromosome 12p (ETV6-AML), Fc fragment of IgA receptor (FCAR), Fc receptor-like 5 (FCRL5), Fms-like tyrosine kinase 3 (FLT3), Folate receptor beta, Fos-related antigen 1, Fucosyl GM1, G protein-coupled receptor 20 (GPR20), G protein-coupled receptor class C group 5, member D (GPRC5D), ganglioside GD3, ganglioside GM3, glycoceramide (GloboH), Glypican-3 (GPC3), Hepatitis A virus cellular receptor 1 (HAVCR1), hexasaccharide portion of globoH, high molecular weight-melanoma-associated antigen (HMWMAA), human Telomerase reverse transcriptase (hTERT), interleukin 11 receptor alpha (IL-11Ra), KIT (CD117), leukocyte-associated immunoglobulin-like receptor 1 (LAIR1), leukocyte immunoglobulin-like receptor subfamily A member 2 (LILRA2), Lewis(Y) antigen, lymphocyte antigen 6 complex, locus K 9 (LY6K), lymphocyte antigen 75 (LY75), lymphocyte-specific protein tyrosine kinase (LCK), mammary gland differentiation antigen (NY-BR-1), melanoma cancer testis antigen-1 (MAD-CT-1), melanoma cancer testis antigen-2 (MAD-CT-2), melanoma inhibitor of apoptosis (ML-IAP), mucin 1, cell surface associated (MUC1), N-acetyl glucosaminyl-transferase V (NA17), neural cell adhesion molecule (NCAM), o-acetyl-GD2 ganglioside (OAcGD2), olfactory receptor 51E2 (OR51E2), p53 mutant, paired box protein Pax-3 (PAX3), paired box protein Pax-5 (PAX5), pannexin 3 (PANX3), placenta-specific 1 (PLAC1), platelet-derived growth factor receptor beta (PDGFR-beta), Polysialic acid, proacrosin binding protein sp32 (OY-TES 1), prostate stem cell antigen (PSCA), Protease Serine 21 (PRSS21), Proteasome (Prosome, Macropain) Subunit, Beta Type, 9 (LMP2), Ras Homolog Family Member C (RhoC), sarcoma translocation breakpoints, sialyl Lewis adhesion molecule (sLe), sperm protein 17 (SPA17), squamous cell carcinoma antigen recognized by T cells 3 (SART3), stage-specific embryonic antigen-4 (SSEA-4), synovial sarcoma, X breakpoint 2 (SSX2), TCR gamma alternate reading frame protein (TARP), TGS5, thyroid stimulating hormone receptor (TSHR), Tn antigen (Tn Ag), tumor endothelial marker 1 (T_{EM}1/CD248), tumor endothelial marker 7-related (T_{EM}7R), uroplakin 2 (UPK2), vascular endothelial growth factor receptor 2 (VEGFR2), v-myc avian myelocytomatosis viral oncogene neuroblastoma derived homolog (MYCN), Wilms tumor protein (WT1), and X Antigen Family, Member 1A (XAGE1), or a fragment or variant thereof.

Additional antigens that may be targeted by the modified cells described herein include, but are not limited to, carbonic anhydrase EX, alpha-fetoprotein, A3, antigen specific for A33 antibody, Ba 733, BrE3-antigen, CA125, CD1, CD1a, CD3, CD5, CD15, CD16, CD19, CD20, CD21, CD22, CD23, CD25, CD30, CD33, CD38, CD45, CD74, CD80, CD123, CD138, Fms related receptor tyrosine kinase 3 (FLT3) or CD135, colon-specific antigen-p (CSAp), CEA (CEACAM5), CEACAM6, CSAp, EGFR, EGP-I, EGP-2, Ep-CAM, EphA1, EphA3, EphA4, EphA5, EphA6, EphA7, EphA8, EphA10, EphB1, EphB2, EphB3, EphB4, EphB6, FIt-I, Flt-3, folate receptor, HLA-DR, human chorionic gonadotropin (HCG) and its subunits, hypoxia inducible factor (HIF-I), Ia, IL-2, IL-6, IL-8, insulin growth factor-1 (IGF-I), KC4-antigen, KS-1-antigen, KS1-4, Le-Y, macrophage inhibition factor (MIF), MAGE, MUC1, MUC2, MUC3, MUC4, NCA66, NCA95, NCA90, antigen specific for PAM-4 antibody, placental growth factor, p53, prostatic acid phosphatase, PSA, PSMA, RS5, S100, TAC, TAG-72, tenascin, TRAIL receptors, Tn antigen, Thomson-Friedenreich antigens, tumor necrosis antigens, VEGF, ED-B fibronectin, 17-lA-antigen, an angiogenesis marker, an oncogene marker or an oncogene product.

In particular embodiments, the tumor antigen that is targeted by the modified cells described herein is BCMA, GPRC5D, CD79, KLK2, CD19, CD30, CD33, CD123, hK2, FLT3, CD20, CD22, KRASG12D, p53, BRAC1 or PSMA.

In one embodiment, the CAR that specifically binds to BCMA comprises an extracellular target-binding domain that comprises the amino acid sequence of SEQ ID NO: 2, or a sequence having at least 70%, 75%, 80%, 85%, 90%, 91%, 92%, 93%, 94%, 95%, 96%, 97%, 98%, or 99% identity to SEQ ID NO: 2.

In one embodiment, the CAR that specifically binds to BCMA comprises an extracellular target-binding domain that comprises the amino acid sequence of SEQ ID NO: 3, or a sequence having at least 70%, 75%, 80%, 85%, 90%, 91%, 92%, 93%, 94%, 95%, 96%, 97%, 98%, or 99% identity to SEQ ID NO: 3.

In one embodiment, the CAR that specifically binds to BCMA comprises an amino acid sequence of SEQ ID NO: 11, or a sequence having at least 70%, 75%, 80%, 85%, 90%, 91%, 92%, 93%, 94%, 95%, 96%, 97%, 98%, or 99% identity to SEQ ID NO: 11.

In one embodiment, the CAR that specifically binds to BCMA comprises an extracellular target-binding domain that comprises the amino acid sequence of SEQ ID NO: 12, or a sequence having at least 70%, 75%, 80%, 85%, 90%, 91%, 92%, 93%, 94%, 95%, 96%, 97%, 98%, or 99% identity to SEQ ID NO: 12.

An infectious antigen may be a viral antigen, a bacterial antigen, a fungal antigen, a parasite antigen, or a prion antigen, or the like. Infectious antigens include the intact microorganism (e.g., virus, bacterium, fungus) as well as natural isolates and fragments or derivatives thereof and also synthetic or recombinant compounds which are identical to or similar to natural microorganism antigens and induce an immune response specific for that microorganism (e.g., virus, bacterium, fungus). A compound is similar to a natural microorganism antigen if it induces an immune response (humoral and/or cellular) to a natural microorganism antigen. Such antigens are used routinely in the art and are well known to the skilled artisan.

An infectious antigen may be an infectious virus or derived from an infectious virus. Non-limiting examples of infectious viruses that have been found in humans include but are not limited to: Adenoviridae (most adenoviruses); Arena viridae (hemorrhagic fever viruses); Birnaviridae; Bungaviridae (e.g., Hantaan viruses, bunga viruses, phleboviruses and Nairo viruses); Calciviridae (e.g., strains that cause gastroenteritis); Coronoviridae (e.g., coronaviruses); Filoviridae (e.g., ebola viruses); Flaviridae (e.g., dengue viruses, encephalitis viruses, yellow fever viruses); Hepadnaviridae (Hepatitis B virus); Herpesviridae (herpes simplex virus (HSV) 1 and 2, varicella zoster virus, cytomegalovirus (CMV), herpes virus); Iridoviridae (e.g., African swine fever virus); Norwalk and related viruses, and astroviruses; Orthomyxoviridae (e.g., influenza viruses); Papovaviridae (papilloma viruses, polyoma viruses); Paramyxoviridae (e.g., parainfluenza viruses, mumps virus, measles virus, respiratory syncytial virus); Parvovirida (parvoviruses); Picornaviridae (e.g., polio viruses, hepatitis A virus; enteroviruses, human Coxsackie viruses, rhinoviruses, echoviruses); Poxviridae (variola viruses, vaccinia viruses, pox viruses); Reoviridae (e.g., reoviruses, orbiviurses and rotaviruses); Retroviridae (e.g., human immunodeficiency viruses, such as HIV-1 (also referred to as HTLV-III, LAV or HTLV-III/LAV, or HIV-III); and other isolates, such as HIV-LP); Rhabdoviradae (e.g., vesicular stomatitis viruses, rabies viruses); Togaviridae (e.g., equine encephalitis viruses, rubella viruses); and unclassified viruses (e.g., the etiological agents of Spongiform encephalopathies, the agent of delta hepatitis, the agents of non-A, non-B hepatitis (i.e. Hepatitis C)).

An infectious antigen may be an infectious bacterium or derived from an infectious bacterium. Both gram negative and gram positive bacteria can serve as antigens in vertebrate animals. Such gram positive bacteria include, but are not limited to, Pasteurella species, Staphylococci species and Streptococcus species. Grain negative bacteria include, but are not limited to, Escherichia coli, Pseudomonas species, and Salmonella species. Non-limiting examples of infectious bacteria include but are not limited to: Actinomyces israelli, Bacillus antracis, Bacteroides sp., Borelia burgdorferi, Chlamydia, Clostridium perfringers, Clostridium tetani, Corynebacterium diphtheriae, Corynebacterium sp., Enterobacter aerogenes, Enterococcus sp., Erysipelothrix rhusiopathiae, Fusobacterium nucleatum, Haemophilus influenzae, Helicobacter pyloris, Klebsiella pneumoniae, Legionella pneumophilia, Leptospira, Listeria monocytogenes, Mycobacteria sps. (e.g., M tuberculosis, M avium, M gordonae, M intracellulare, M kansaii), Neisseria gonorrhoeae, Neisseria meningitidis, Pasteurella multocida, pathogenic Campylobacter sp., Rickettsia, Staphylococcus aureus, Streptobacillus monihformis, Streptococcus (anaerobic sps.), Streptococcus (viridans group), Streptococcus agalactiae (Group B Streptococcus), Streptococcus bovis, Streptococcus faecalis, Streptococcus pneumoniae, Streptococcus pyogenes (Group A Streptococcus), Treponema pallidium, and Treponema pertenue.

An infectious antigen may be or derived from other infectious microorganisms. Non-limiting examples of infectious fungi include: Cryptococcus neoformans, Histoplasma capsulatuin, Coccidioides immitis, Blastomyces dernatitidis, Chlamydia trachomatis and Candida albicans. Other infectious organisms (i.e., protists) include: Plasmodium such as Plasmodium falciparum, Plasmodium malariae, Plasmodium ovale, Plasmodium vivax, Toxoplasma gondii and Shistosoma. Other medically relevant microorganisms have been descried extensively in the literature, e.g., see C. G. A. Thomas, “Medical Microbiology”, Bailliere Tindall, Great Britain 1983, which is hereby incorporated by reference in its entirety.

Other non-limiting examples of infectious antigens include viral antigens such as HIV antigens (e.g., gp120, gp160, p18, Tat, Gag, Pol, Env, Nef), glycoprotein from Herpesvirus, and surface antigen and core antigen from Hepatitis B virus; bacterial antigens such as OspA, OspB and OspC antigens from Borrelia sp; fungal and parasite antigens such as MP65 from Candida albicans and CS protein from Plasmodium sp.

In some embodiments, the CAR or the engineered TCR may be directed against a self antigen. Examples of such antigens include those associated with autoimmune diseases, such as Rheumatoid arthritis (RA), multiple sclerosis (MS), Sjögren's syndrome, sarcoidosis, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Crohn's disease and ulcerative colitis.

In additional embodiments, the methods of the present disclosure may also involve reducing or inhibiting the expression of one or more endogenous T cell receptor (TCR).

Various embodiments of the methods described above involves introducing into the cells one or more polynucleotide/polypeptide agents (e.g., CARs or engineered TCR). The polynucleotides and/or polypeptides described in the present invention may be introduced into the cell via viral, non-viral gene delivery methods, or a physical method. Suitable methods for polynucleotide and/or polypeptide delivery for use the methods of the present invention include any method known by those of skill in the art, by which a polynucleotide and/or polypeptide can be introduced into an organelle, cell, tissue or organism. The polynucleotide and/or polypeptide transfer may be carried out in vitro, ex vivo, or in vivo.

In various embodiment, polypeptides or polynucleotides are introduced into the cells using a physical method. Suitable physical methods include, but are not limited to, electroporation, direct injection (e.g., microinjection), magnetofection, ultrasound, a ballistic or hydrodynamic method, or a combination thereof.

Electroporation is a method for polynucleotide and/or polypeptide delivery. See e.g., Potter et al., (1984) Proc. Nat'l Acad. Sci. USA, 81, 7161-7165 and Tur-Kaspa et al., (1986) Mol. Cell Biol., 6, 716-718, both of which are incorporated herein in their entirety for all purposes. Electroporation involves the exposure of a suspension of cells and DNA to a high-voltage electric discharge. In some embodiments, cell wall-degrading enzymes, such as pectin-degrading enzymes, can be employed to render the cells more susceptible to genetic modification by electroporation than untreated cells. See e.g., U.S. Pat. No. 5,384,253, incorporated herein by reference in its entirety for all purposes.

When CRISPR/Cas nucleases are used, one or more CRISPR/Cas nucleases and one or more gRNAs may be assembled to form one or more ribonucleoprotein (RNP) complexes which are then introduced into the cells by electroporation.

Methods of electroporation for use with this invention include, for example, Sardesai, N. Y., and Weiner, D. B., Current Opinion in Immunotherapy 23:421-9 (2011) and Ferraro, B. et al., Human Vaccines 7:120-127 (2011), both of which are hereby incorporated by reference in their entirety for all purposes.

Another physical method for polynucleotide and/or polypeptide transfer includes injection. In some embodiments, a polypeptide, a polynucleotide, or a vector may be delivered to a cell, tissue, or organism via one or more injections (e.g., a needle injection). Non-limiting methods of injection include injection of a composition (e.g., a saline based composition). Polynucleotides and/or polynucleotides can also be introduced by direct microinjection. Non-limiting sites of injection include, subcutaneous, intradermal, intramuscular, intranodal (allows for direct delivery of antigen to lymphoid tissues), intravenous, intraprotatic, intratumor, intralymphatic (allows direct administration of dendritic cells) and intraperitoneal. It is understood that proper site of injection preparation is necessary (e.g., shaving of the site of injection to observe proper needle placement).

In some embodiments, polynucleotides and/or polypeptides described in the present invention are introduced into cells by pinocytosis induced by hypertonicity or hypotonicity. For example, the cells maybe placed into a buffer that has either a higher or lower salt concentration than normal saline. This may activate an active uptake mechanism in the cells where they engulf the extracellular environment. Various chemicals can be used to enhance and modify this process. It may not require any special machinery. Exemplary ways that pinocytosis can be used for transduction are described in the art. See e.g., WO2017093326A1, which is hereby incorporated by reference in its entirety for all purposes.

In various embodiments, polynucleotides and/or polypeptides described in the present invention are introduced into cells via a vector. The vector may be a viral vector or a non-viral vector.

In some embodiments, the vector is a viral vector. Suitable viral vectors that can be used in the present invention include, but are not limited to, a retroviral vector, a lentiviral vector, an adenoviral vector, an adeno-associated viral (AAV) vector, an alphaviral vector, vaccinia virus vector, a herpes simplex virus vector, or a baculoviral vector. In one specific embodiment, the viral vector is a lentiviral vector. In one specific embodiment, the viral vector is a retroviral vector. In some embodiments, cells are transduced via retroviral transduction. References describing retroviral transduction of genes are Anderson et al., U.S. Pat. No. 5,399,346; Mann et al., Cell 33:153 (1983); Temin et al., U.S. Pat. No. 4,650,764; Temin et al., U.S. Pat. No. 4,980,289; Markowitz et al., J. Virol. 62:1120 (1988); Temin et al., U.S. Pat. No. 5,124,263; International Patent Publication No. WO 95/07358, published Mar. 16, 1995, by Dougherty et al.; and Kuo et al., Blood 82:845 (1993), each of which is incorporated herein by reference in its entirety.

In some embodiments, the vector is a non-viral vector. Non-limiting examples of non-viral vectors useful in the methods of the present invention include a plasmid or a transposon.

Nucleic acid vaccines may also be used to transfer polynucleotides into the cells. Such vaccines include, but are not limited to non-viral polynucleotide vectors, “naked” DNA and RNA, and viral vectors. Methods of genetically modifying cells with these vaccines, and for optimizing the expression of genes included in these vaccines are known to those of skill in the art.

In some embodiments, polynucleotides and/or polypeptides may be introduced into the cells using a nanoparticle, a polymer, a dendrimer, a liposome, and a polyethylenimine (PEI) particle. In some embodiments, polypeptides (e.g., CRISPR/Cas nucleases) are introduced into the cells as a soluble protein or ribonucleoprotein.

Additional methods of polynucleotide and/or polypeptide transfer include liposome-mediated transfection (e.g., polynucleotide entrapped in a lipid complex suspended in an excess of aqueous solution. See e.g., Ghosh and Bachhawat, (1991) In: Liver Diseases, Targeted Diagnosis and Therapy Using Specific Receptors and Ligands. pp. 87-104). Also contemplated is a polynucleotide and/or polypeptide complexed with Lipofectamine, or Superfect); DEAE-dextran (e.g., a polynucleotide is delivered into a cell using DEAE-dextran followed by polyethylene glycol. See e.g., Gopal, T. V., Mol Cell Biol. 1985 May; 5(5):1188-90); calcium phosphate (e.g., polynucleotide is introduced to the cells using calcium phosphate precipitation. See e.g., Graham and van der Eb, (1973) Virology, 52, 456-467; Chen and Okayama, Mol. Cell Biol., 7(8):2745-2752, 1987), and Rippe et al., Mol. Cell Biol., 10:689-695, 1990); sonication loading (introduction of a polynucleotide by direct sonic loading. See e.g., Fechheimer et al., (1987) Proc. Nat'l Acad Sci. USA, 84, 8463-8467); microprojectile bombardment (e.g., one or more particles may be coated with at least one polynucleotide and/or polypeptide and delivered into cells by a propelling force. See e.g., U.S. Pat. Nos. 5,550,318; 5,538,880; 5,610,042; and PCT Application WO 94/09699; Klein et al., (1987) Nature, 327, 70-73, Yang et al., (1990) Proc. Nat'l Acad Sci. USA, 87, 9568-9572); and receptor-mediated transfection (e.g., selective uptake of macromolecules by receptor-mediated endocytosis that will be occurring in a target cell using cell type-specific distribution of various receptors. See e.g., Wu and Wu, (1987) J. Biol. Chem., 262, 4429-4432; Wagner et al., Proc. Natl. Acad Sci. USA, 87(9):3410-3414, 1990; Perales et al., Proc. Natl. Acad Sci. USA, 91:4086-4090, 1994; Myers, EPO 0273085; Wu and Wu, Adv. Drug Delivery Rev., 12:159-167, 1993; Nicolau et al., (1987) Methods Enzymol., 149, 157-176), each reference cited here is incorporated by reference in their entirety for all purposes.

It should be recognized that in the case of CRISPR/Cas nucleases, the Cas protein (e.g., Cas9, Cas12a) and the gRNA need not to be delivered using the same method. In some embodiments, the Cas protein (e.g., Cas9, Cas12a) and the gRNA are delivered using the same method. For example, both the Cas protein (e.g., Cas9, Cas12a) and the gRNA can be introduced into the cells via electroporation or in the same vector. In some embodiments, the Cas protein (e.g., Cas9, Cas12a) and the gRNA are delivered using different methods. For example, the Cas protein (e.g., Cas9, Cas12a) is introduced into the cells via electroporation and the gRNA is delivered in viral vector. As another example, the Cas protein (e.g., Cas9, Cas12a) and the gRNA are delivered in separate vectors.

### Stimulation/Activation of T Cells

In order to reach sufficient therapeutic doses of cell compositions, T cells may be subjected to one or more rounds of stimulation/activation. Cells may be activated and/or expanded ex vivo before, after and/or during the genetic modification step.

In some embodiments, stimulation/activation of T cells may be performed in the presence of one or more cytokines comprising IL-7, IL-15 and/or IL-21. In some embodiments, stimulation/activation of T cells may be performed in the presence of IL-7. In some embodiments, stimulation/activation of T cells may be performed in the presence of IL-7 and IL-15. In some embodiments, stimulation/activation of T cells may be performed in the presence of IL-7 and IL-21. In some embodiments, stimulation/activation of T cells may be performed in the presence of IL-7, IL-15 and IL-21.

In some embodiments, stimulation/activation of T cells may be performed in the absence of IL-2.

In some embodiments, a method described herein comprises stimulating T cells to become activated in the presence of one or more stimulatory signals or agents (e.g., compound, small molecule, e.g., small organic molecule, nucleic acid, polypeptide, or a fragment, isoform, variant, analog, or derivative thereof). In some embodiments, a method described herein comprises stimulating T cells to become activated and to proliferate in the presence of one or more stimulatory signals or agents.

T cells can be activated by inducing a change in their biologic state by which the cells express activation markers, produce cytokines, proliferate and/or become cytotoxic to target cells. All these changes can be produced by primary stimulatory signals. Co-stimulatory signals amplify the magnitude of the primary signals and suppress cell death following initial stimulation resulting in a more durable activation state and thus a higher cytotoxic capacity.

T cells can be activated generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety for all purposes.

In some embodiments, alpha-beta T cells can be activated using CD3/CD28 stimulation. As an example, alpha-beta T cells may be stimulated using anti-CD3 and anti-CD28 antibodies.

In some embodiments, gamma-delta T cells can be activated by zoledronate and/or an agent (e.g., an antibody) that binds the gamma-delta TCR. IL-2 and IL-15 can also be used to expand gamma-delta T cells.

In some embodiments, T cells can be activated by binding to an agent that activates CD3ζ.

In other embodiments, a CD2-binding agent may be used to provide a primary stimulation signal to the T cells. For example, and not by limitation, CD2 agents include, but are not limited to, CD2 ligands and anti-CD2 antibodies, e.g., the Tl 1.3 antibody in combination with the Tl 1.1 or Tl 1.2 antibody (Meuer, S. C. et al. (1984) Cell 36:897-906, which is incorporated herein by reference in its entirety) and the 9.6 antibody (which recognizes the same epitope as TI 1.1) in combination with the 9-1 antibody (Yang, S. Y. et al. (1986) J. Immunol. 137:1097-1100, which is incorporated herein by reference in its entirety). Other antibodies which bind to the same epitopes as any of the above described antibodies can also be used.

In some embodiments, T cells are activated by administering phorbol myristate acetate (PMA) and ionomycine. In some embodiments, the T cells are activated by administering an appropriate antigen that induces activation and then expansion. In some embodiments, PMA, ionomycin, and/or appropriate antigen are administered with CD3 induce activation and/or expansion.

In general, the activating agents used in the present invention includes, but is not limited to, an antibody, a fragment thereof and a proteinaceous binding molecule with antibody-like functions. Examples of (recombinant) antibody fragments are Fab fragments, Fv fragments, single-chain Fv fragments (scFv), a divalent antibody fragment such as an (Fab)2′-fragment, diabodies, triabodies (Iliades, P., et al., FEBS Lett (1997) 409, 437-441, which is incorporated herein by reference in its entirety), decabodies (Stone, E., et al., Journal of Immunological Methods (2007) 318, 88-94, which is incorporated herein by reference in its entirety) and other domain antibodies (Holt, L. J., et al., Trends Biotechnol. (2003), 21, 11, 484-490, which is incorporated herein by reference in its entirety). The divalent antibody fragment may be an (Fab)2′-fragment, or a divalent single-chain Fv fragment while the monovalent antibody fragment may be selected from the group consisting of a Fab fragment, a Fv fragment, and a single-chain Fv fragment (scFv).

In some embodiments, one or more binding sites of the CD3 (agents may be a bivalent proteinaceous artificial binding molecule such as a dimeric lipocalin mutein (i.e., duocalin). In some embodiments the receptor binding reagent may have a single second binding site, (i.e., monovalent). Examples of monovalent agents include, but are not limited to, a monovalent antibody fragment, a proteinaceous binding molecule with antibody-like binding properties or an MHC molecule. Examples of monovalent antibody fragments include, but are not limited to a Fab fragment, a Fv fragment, and a single-chain Fv fragment (scFv), including a divalent single-chain Fv fragment.

The agent that specifically binds CD3 includes, but is not limited to, an anti-CD3-antibody, a divalent antibody fragment of an anti-CD3 antibody, a monovalent antibody fragment of an anti-CD3-antibody, and a proteinaceous CD3-binding molecule with antibody-like binding properties. A proteinaceous CD3-binding molecule with antibody-like binding properties can be an aptamer, a mutein based on a polypeptide of the lipocalin family, a glubody, a protein based on the ankyrin scaffold, a protein based on the crystalline scaffold, an adnectin, and an avimer. It also can be coupled to a bead.

In some embodiments, the activating agent (e.g., CD3-binding agents) can be present in a concentration of about 0.1 to about 10 μg/ml. In some embodiments, the activating agent (e.g., CD3-binding agents) can be present in a concentration of about 0.2 μg/ml to about 9 μg/ml, about 0.3 μg/ml to about 8 μg/ml, about 0.4 μg/ml to about 7 μg/ml, about 0.5 μg/ml to about 6 μg/ml, about 0.6 μg/ml to about 5 μg/ml, about 0.7 μg/ml to about 4 μg/ml, about 0.8 μg/ml to about 3 g/ml, or about 0.9 μg/ml to about 2 μg/ml. In some embodiments, the activating agent (e.g., CD3-binding agents) is administered at a concentration of about 0.1 μg/ml, about 0.2 μg/ml, about 0.3 g/ml, about 0.4 μg/ml, about 0.5 μg/ml, about 0.6 μg/ml, about 0.7 μg/ml, about 0.8 μg/ml, about 0.9 μg/ml, about 1 μg/ml, about 2 μg/ml, about 3 μg/ml, about 4 μg/ml, about 5 μg/ml, about 6 g/ml, about 7 μg/ml, about 8 μg/ml, about 9 μg/ml, or about 10 μg/ml. In some embodiments, the CD3-binding agents can be present in a concentration of 1 μg/ml.

In some embodiments, the activating agent is attached to a solid support such as, but not limited to, a bead, an absorbent polymer present in culture plate or well or other matrices such as, but not limited to, Sepharose or glass; may be expressed (such as in native or recombinant forms) on cell surface of natural or recombinant cell line by means known to those skilled in the art.

### Expansion/Proliferation of T Cells

After T cells are activated and transduced, the cells can be cultured to proliferate. T cells may be cultured for at least 1, 2, 3, 4, 5, 6, 7, 8, 9, 10, 11, 12, 13, 14, 15, 16, 17, 18, 19, or 20 days, at least 1 week or 2 weeks, at least 1, 2, 3, 4, 5, or 6 months or more with 1, 2, 3, 4, 5, 6, 7, 8, 9, or 10 or more rounds of expansion. In some embodiments, T cells are cultured for 1-20 days, 1-18 days, 1-14 days, 3-20 days, 3-18 days, 3-14 days, 5-20 days, 5-18 days, 5-14 days, 7-20 days, 7-18 days, 7-16 days, 7-15 days, 7-14 days, 8-20 days, 8-18 days, 8-16 days, 8-15 days, 8-14 days, 8-13 days, 8-12 days, 9-20 days, 9-18 days, 9-16 days, 9-15 days, 9-14 days, 9-13 days, 9-12 days, 10-20 days, 10-18 days, 10-16 days, 10-15 days, or 10-14 days. In some embodiments, T cells are cultured for 8-14 days. In one embodiment, T cells are cultured for 14 days.

According to the present disclosure, stem-cell like memory T (T_{SCM}) cells can be enriched by culturing the T cells with interleukin(s) such as IL-7, IL-15, and/or IL-21.

In some embodiments, stem-cell like memory T (T_{SCM}) cells can be enriched by culturing the T cells in the presence of IL-7. In some embodiments, stem-cell like memory T (T_{SCM}) cells can be enriched by culturing the T cells in the presence of IL-7 and IL-15. In some embodiments, stem-cell like memory T (T_{SCM}) cells can be enriched by culturing the T cells in the presence of IL-7 and IL-21. In some embodiments, stem-cell like memory T (T_{SCM}) cells can be enriched by culturing the T cells in the presence of IL-7, IL-15 and IL-21. In some embodiments, stem-cell like memory T (T_{SCM}) cells can be enriched by culturing the T cells in the presence of IL-7, IL-15 and IL-21.

In some embodiments, interleukin(s) used to enrich stem-cell like memory T (T_{SCM}) cells do not include IL-2.

In some embodiments, the agent(s) used for expansion (e.g., IL-7, IL-15, IL-21) are administered at about 1 ng/ml to about 20 ng/ml. In some embodiments, the agent(s) used for expansion (e.g., IL-7, IL-15, IL-21) are administered at about 2 ng/ml to about 20 ng/ml, 5 ng/ml to about 20 ng/ml, 5 ng/ml to about 18 ng/ml, 5 ng/ml to about 15 ng/ml, 5 ng/ml to about 12 ng/ml, 7 ng/ml to about 20 ng/ml, 7 ng/ml to about 18 ng/ml, 7 ng/ml to about 15 ng/ml, 5.5 ng/ml to about 9.5 ng/ml, about 6 ng/ml to about 9 ng/ml, about 6.5 ng/ml to about 12 ng/ml, or about 9 ng/ml to about 12 ng/ml. In some embodiments, the agent(s) used for expansion (e.g., IL-7, IL-15, IL-21) are administered at about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml or about 15 ng/ml.

Other illustrative examples for agents that may be used for the expansion of T cells are agents that bind to CD8, CD45 or CD90, such as αCD8, αCD45 or αCD90 antibodies. Illustrative examples of T cell population including antigen-specific T cells, T helper cells, cytotoxic T cells, memory T cell (an illustrative example of memory T-cells are CD62L⁺CD8⁺ specific central memory T cells) or regulatory T cells (an illustrative example of Treg are CD4+CD25+CD45RA⁺ Treg cells).

Additional agents that can be used to expand T lymphocytes includes methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety for all purposes.

Conditions appropriate for T cell culture include an appropriate media (e.g., Minimal Essential Media (MEM), RPMI Media 1640, Lonza RPMI 1640, Advanced RPMI, Clicks, AIM-V, DMEM, a-MEM, F-12, TexMACS, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion).

Examples of other additives for T cell expansion include, but are not limited to, surfactant, piasmanate, pH buffers such as HEPES, and reducing agents such as N-acetyl-cysteine and 2-mercaptoethanol, Antibiotics (e.g., penicillin and streptomycin), are included only in experimental cultures, not in cultures of cells that are to be infused into a subject. The target cells are maintained under conditions necessary to support growth, for example, an appropriate temperature (e.g., 37° C.) and atmosphere (e.g., air plus 5% C02).

In some embodiments wherein the cells are iPSC derived cells, the modified cells are selected for individual clones to make a master cell bank.

In some embodiments, the percentage of T_{SCM} cells are determined in the population of T cells after cell expansion. In some embodiments, the percentage of T_{SCM} cells in the population of T cells is at least about 30%, 35%, 40%, 45%, 50%, 55%, 60%, 65%, 70%, 75%, 80%, 85%, 90% or 95% after cell expansion. In one embodiment, the percentage of T_{SCM} cells in the population of T cells is about 30%-90%, 40%-80%, 50%-60%, 50%-70%, 50%-80%, 60%-70%, or 60%-80% after cell expansion.

### Compositions of the Invention

In one aspect, the present disclosure provides a population of the T cells comprising the stem-cell like memory T (T_{SCM}) cells prepared according to the method described herein. The cells may have been genetically modified to express one or more CARs or engineered TCRs.

In another aspect, the present disclosure also provides a pharmaceutical composition comprising the population of T cells comprising the stem-cell like memory T (T_{SCM}) cells and optionally a pharmaceutically acceptable carrier and/or excipient. Examples of pharmaceutical carriers include but are not limited to sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions.

Compositions comprising modified cells described herein may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

Compositions comprising modified cells described herein may comprise one or more of the following: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is preferably sterile.

In some embodiments, compositions are formulated for parenteral administration, e.g., intravascular (intravenous or intraarterial), intraperitoneal, intratumoral, intraventricular, intrapleural or intramuscular administration. In some embodiments, the composition is reconstituted from a lyophilized preparation prior to administration.

In some embodiments, the modified cells may be mixed with substances that adhere or penetrate prior to their administration, e.g., but not limited to, nanoparticles.

### Treatment Methods

In another aspect, the present disclosure provides a method of transplantation in a subject in need thereof, the method including administering to the subject an effective amount of the population of T cells comprising the stem-cell like memory T (T_{SCM}) cells as described herein or the pharmaceutical composition described herein.

In another aspect, the present disclosure provides a method of treating a disease or disorder in a subject in need thereof, the method including administering to the subject an effective amount of the population of cells comprising the stem-cell like memory T (T_{SCM}) cells as described herein or the pharmaceutical composition as described herein.

In another aspect, the present disclosure provides the population of T cells as described herein, or the pharmaceutical composition for use in medicine. In some embodiments, the present disclosure provides the population of T cells as described herein, or the pharmaceutical composition for use in treating a disease or disorder, for example, but are not limited to, a cancer, an autoimmune disease, or an infection.

In some embodiments, the methods for manufacturing the population of T cells or pharmaceutical compositions are performed in vitro or ex vivo.

In some embodiments, the population of cells or the cells in the pharmaceutical composition are allogeneic with respect to a subject to be administered the population of cells or the pharmaceutical composition. In some embodiments, the population of cells or the cells in the pharmaceutical composition are autologous with respect to a subject to be administered the population of cells or the pharmaceutical composition.

Diseases or disorders that can be treated using the methods and/or compositions of the present disclosure include, but are not limited to, a cancer, an autoimmune disease, or an infection.

In some embodiments, the disease or disorder that can be treated with a method described herein is a cancer. The terms “cancer” and “cancerous” refer to or describe the physiological condition in mammals that is typically characterized by unregulated cell growth. The term “cancer” includes, for example, the soft tissue tumors (e.g., lymphomas), and tumors of the blood and blood-forming organs (e.g., leukemias), and solid tumors, which is one that grows in an anatomical site outside the bloodstream (e.g., carcinomas). Examples of cancer include, but are not limited to, carcinoma, lymphoma, blastoma, sarcoma (e.g., osteosarcoma or rhabdomyosarcoma), and leukemia or lymphoid malignancies. More particular examples of such cancers include squamous cell cancer (e.g., epithelial squamous cell cancer), adenosquamous cell carcinoma, lung cancer (e.g., including small-cell lung cancer, non-small cell lung cancer, adenocarcinoma of the lung, squamous carcinoma of the lung, bronchogenic carcinoma, Lewis lung carcinoma, lung neuroendocrine tumors, typical carcinoid, atypical carcinoid, and large cell neuroendocrine carcinoma), cancer of the peritoneum, hepatocellular cancer, gastric or stomach cancer (e.g., including gastrointestinal cancer, gastrointestinal stromal tumor pancreatic cancer, pancreatic adenocarcinoma, intraductal papillary mucinous neoplasm (JPMN), Islet cell tumors), cervical cancer (including, but not limited to, cervical adenocarcinoma), ovarian cancer (including, but not limited to, cystadenocarcinoma, ovarian embryonal carcinoma, ovarian adenocarcinoma, ovarian clear cell carcinoma, ovarian serous cystadenoma), liver cancer, bladder cancer, cancer of the urinary tract, hepatoma, breast cancer (including, but not limited to, adenocarcinoma of the breast, papillary carcinoma of the breast, mammary cancer, medullary carcinoma of the breast), colon cancer (including, but not limited to colon adenocarcinoma), colorectal cancer (including, but not limited to, rectal cancer, colorectal adenocarcinoma), endometrial cancer (including, but not limited to, uterine cancer, uterine sarcoma), salivary gland carcinoma, kidney or renal cancer (including, but not limited to, nephroblastoma or Wilms' tumor, renal cell carcinoma), prostate cancer (including, but not limited to, prostate adenocarcinoma), vulval cancer, thyroid cancer, hepatic carcinoma, anal carcinoma, penile carcinoma, skin cancer (including, but not limited to, primary or metastatic melanoma, squamous cell carcinoma, keratoacanthoma, basal cell carcinoma), multiple myeloma (including, but not limited to, smoldering multiple myeloma) and acute lymphocytic leukemia (ALL) (including, but not limited to, B-cell ALL, T-cell ALL), acute myelocytic leukemia (AML) (e.g., B-cell AML, T-cell AML), chronic myelocytic leukemia (CMNL) (e.g., B-cell CML, T-cell CML), and chronic lymphocytic leukemia (CLL) (e.g., B-cell CLL, T-cell CLL), lymphoma such as Hodgkin lymphoma (HL) (including, but not limited to, B-cell HL, T-cell HL) and non-Hodgkin lymphoma (NIL) (e.g., B-cell NIL such as diffuse large cell lymphoma (DLCL) (e.g., diffuse large B-cell lymphoma (DLIBCL)), follicular lymphoma, chronic lymphocytic leukemia/small lymphocytic lymphoma (CLL/SLL), mantle cell lymphoma (MCL), marginal zone B-cell lymphomas (including, but not limited to, mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (including, but not limited to, Waldenstrom's macro globulinemia), immunoblastic large cell lymphoma, hairy cell leukemia (HCL), precursor B-lymphoblastic lymphoma and primary central nervous system (CNS) lymphoma, T-cell NHL such as precursor T-lymphoblastic lymphoma/leukemia, peripheral T-cell lymphoma (PTCL) (e.g. cutaneous T-cell lymphoma (CTCL) including, but not limited to, mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma, a mixture of one or more leukemia/lymphoma as described above, brain (e.g., high grade glioma, diffuse pontine glioma, ependymoma, neuroblastoma, meningioma, astrocytoma, oligodendroglioma; medulloblastoma, or glioblastoma), as well as head and neck cancer (including, but not limited to, head and neck squamous cell carcinoma), biliary cancer (including, but not limited to, cholangiocarcinoma), bronchus cancer, chordoma, choriocarcinoma, epithelial carcinoma, endothelial sarcoma (including, but not limited to, Kaposi's sarcoma, multiple idiopathic hemorrhagic sarcoma), esophageal cancer (including, but not limited to, adenocarcinoma of the esophagus, Barrett's adenocarinoma), Ewing sarcoma, heavy chain disease (including, but not limited to, alpha chain disease, gamma chain disease, mu chain disease), hematopoietic cancer, immunocytic amyloidosis, monoclonal gammopathy of undetermined significance, myelodysplastic syndromes, myeloproliferative disorder, agnogenic myeloid metaplasia (AMM) or myelofibrosis (MF), chronic idiopathic myelofibrosis, myeloproliferative neoplasms, polycythemia vera, rectum adenocarcinoma, essential thrombocytosis, chronic neutrophilic leukemia, hypereosinophilic syndrome, soft tissue sarcoma (e.g. malignant fibrous histiocytoma (MFH), liposarcoma, malignant peripheral nerve sheath tumor (MPNST), chondrosarcoma, fibrosarcoma, myxosarcoma), and associated metastases. Additional examples of cancer can be found in The Merck Manual of Diagnosis and Therapy, 19th Edition, § on Hematology and Oncology, published by Merck Sharp & Dohme Corp., 2011 (ISBN 978-O-911910-19-3); The Merck Manual of Diagnosis and Therapy, 20th Edition, § on Hematology and Oncology, published by Merck Sharp & Dohme Corp., 2018 (ISBN 978-O-911-91042-1) (2018 digital online edition at internet website of Merck Manuals); and SEER Program Coding and Staging Manual 2016, each of which are incorporated by reference in their entirety for all purposes.

In some embodiments, the cancer is a BCMA-expressing cancer or disorder. In some embodiments, the BCMA-expressing cancer or disorder includes a hematological cancer, such as acute myeloid leukemia (AML) or lymphomas (e.g., multiple myeloma (MM), smoldering multiple myeloma (SMM)).

The compositions and methods described in the present disclosure may be used to treat an infectious disease. Infectious diseases are well known to those skilled in the art, and non-limiting examples include but are not limited to infections of viral etiology such as human immunodeficiency virus (HIV), influenza, Herpes, viral hepatitis, Epstein Bar, polio, viral encephalitis, measles, chicken pox, Papilloma virus, cytomegalovirus, Rabies, Varicella, Yellow fever, West Nile virus, Ebola; infections of bacterial etiology such as pneumonia, tuberculosis, syphilis, Lyme disease, babesiosis; or infections of parasitic etiology such as malaria, trypanosomiasis, leishmaniasis, trichomoniasis, amoebiasis.

The compositions and methods described in the present disclosure may be used to treat an autoimmune disease. Examples of such autoimmune diseases include, but not are limited to, rheumatoid arthritis (RA), multiple sclerosis (MS), Sjögren's syndrome, sarcoidosis, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Crohn's disease and ulcerative coliti.

In some embodiments, the composition is administered in a therapeutically effective amount. The dosages of the composition administered in the methods of the invention will vary widely, depending upon the subject's physical parameters, the frequency of administration, the manner of administration, the clearance rate, and the like. The initial dose may be larger, and might be followed by smaller maintenance doses. The dose may be administered as infrequently as weekly or biweekly, or fractionated into smaller doses and administered daily, semi-weekly, etc., to maintain an effective dosage level. It is contemplated that a variety of doses will be effective to achieve in vivo persistence of the modified cells. It is also contemplated that a variety of doses will be effective to improve in vivo effector function of the modified cells.

In some embodiments, composition comprising the cells generated by the methods described herein may be administered at a dosage of 10² to 10¹⁰ cells/kg body weight, 10⁵ to 10⁹ cells/kg body weight, 10⁵ to 10⁸ cells/kg body weight, 10⁵ to 10⁷ cells/kg body weight, 10⁷ to 10⁹ cells/kg body weight, or 10⁷ to 10⁸ cells/kg body weight, including all integer values within those ranges. The number of cells will depend on the therapeutic use for which the composition is intended for. Therapeutic cells may be administered multiple times at dosages listed above.

The compositions and methods described in the present disclosure may be utilized in conjunction with other types of therapy for cancer, such as chemotherapy, surgery, radiation, gene therapy, and so forth. It is also contemplated that when used to treat various diseases/disorders, the compositions and methods of the present disclosure can be utilized with other therapeutic methods/agents suitable for the same or similar diseases/disorders. Such other therapeutic methods/agents can be co-administered (simultaneously or sequentially) to generate additive or synergistic effects. Suitable therapeutically effective dosages for each agent may be lowered due to the additive action or synergy.

In some embodiments of any of the above therapeutic methods, the method further comprises administering to the subject one or more additional compounds selected from the group consisting of immuno-suppressives, biologicals, probiotics, prebiotics, and cytokines (e.g., IFN or IL-2).

As a non-limiting example, the invention can be combined with other therapies that block inflammation (e.g., via blockage of IL1, INFα/β, IL6, TNF, IL23, etc.).

The methods and compositions of the disclosure can be combined with other immunomodulatory treatments such as, e.g., therapeutic vaccines (including but not limited to GVAX, DC-based vaccines, etc.), checkpoint inhibitors (including but not limited to agents that block CTLA4, PD1, LAG3, TIM3, etc.) or activators (including but not limited to agents that enhance 4-1BB, OX40, etc.). The methods of the disclosure can be also combined with other treatments that possess the ability to modulate NKT function or stability, including but not limited to CD1d, CD1d-fusion proteins, CD1d dimers or larger polymers of CD1d either unloaded or loaded with antigens, CD1d-chimeric antigen receptors (CD1d-CAR), or any other of the five known CD1 isomers existing in humans (CD1a, CD1b, CD1c, CD1e). The methods of the invention can also be combined with other treatments such as midostaurin, enasidenib, or a combination thereof.

Therapeutic methods of the disclosure can be combined with additional, cell therapies, immunotherapies and therapies. For example, when used for treating cancer, the compositions of the invention can be used in combination with conventional cancer therapies, such as, e.g., surgery, radiotherapy, chemotherapy or combinations thereof, depending on type of the tumor, patient condition, other health issues, and a variety of factors. In certain aspects, other therapeutic agents useful for combination cancer therapy with the inhibitors of the invention include anti-angiogenic agents. Many anti-angiogenic agents have been identified and are known in the art, including, e.g., TNP-470, platelet factor 4, thrombospondin-1, tissue inhibitors of metalloproteases (TIMP1 and TIMP2), prolactin (16-Kd fragment), angiostatin (38-Kd fragment of plasminogen), endostatin, bFGF soluble receptor, transforming growth factor beta, interferon alpha, soluble KDR and FLT-1 receptors, placental proliferin-related protein, as well as those listed by Carmeliet and Jain (2000). In one embodiment, the T cells of the invention can be used in combination with a VEGF antagonist or a VEGF receptor antagonist such as anti-VEGF antibodies, VEGF variants, soluble VEGF receptor fragments, aptamers capable of blocking VEGF or VEGFR, neutralizing anti-VEGFR antibodies, inhibitors of VEGFR tyrosine kinases and any combinations thereof (e.g., anti-hVEGF antibody A4.6.1, bevacizumab or ranibizumab).

Non-limiting examples of chemotherapeutic compounds which can be used in combination treatments of the present invention include, for example, aminoglutethimide, amsacrine, anastrozole, asparaginase, azacitidine, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, decitabine, dienestrol, diethylstilbestrol, docetaxel, doxorubicin, epirubicin, estradiol, estramnustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic compounds may be categorized by their mechanism of action into, for example, following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

In various embodiments of the therapeutic methods described herein, the subject is a human. The subject may be a juvenile or an adult, of any age or sex.

### Examples

The following examples are provided to further describe some of the embodiments disclosed herein. The examples are intended to illustrate, not to limit, the disclosed embodiments.

### Example 1. CAR-T Design and Engineering

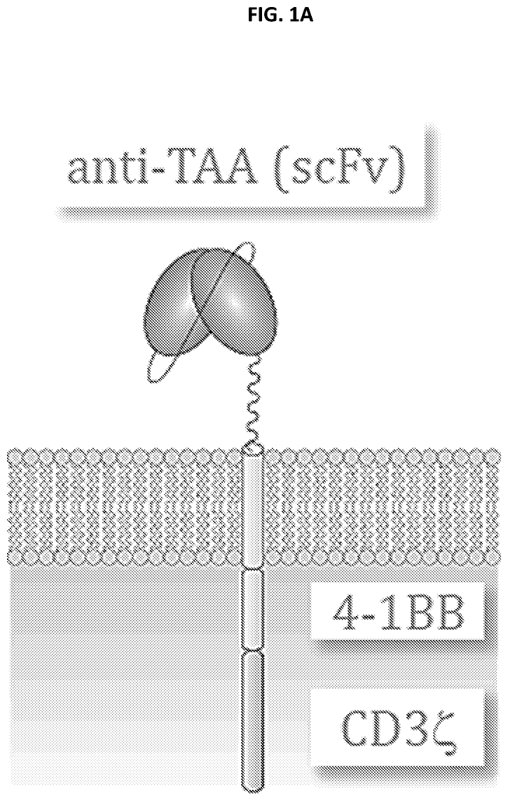

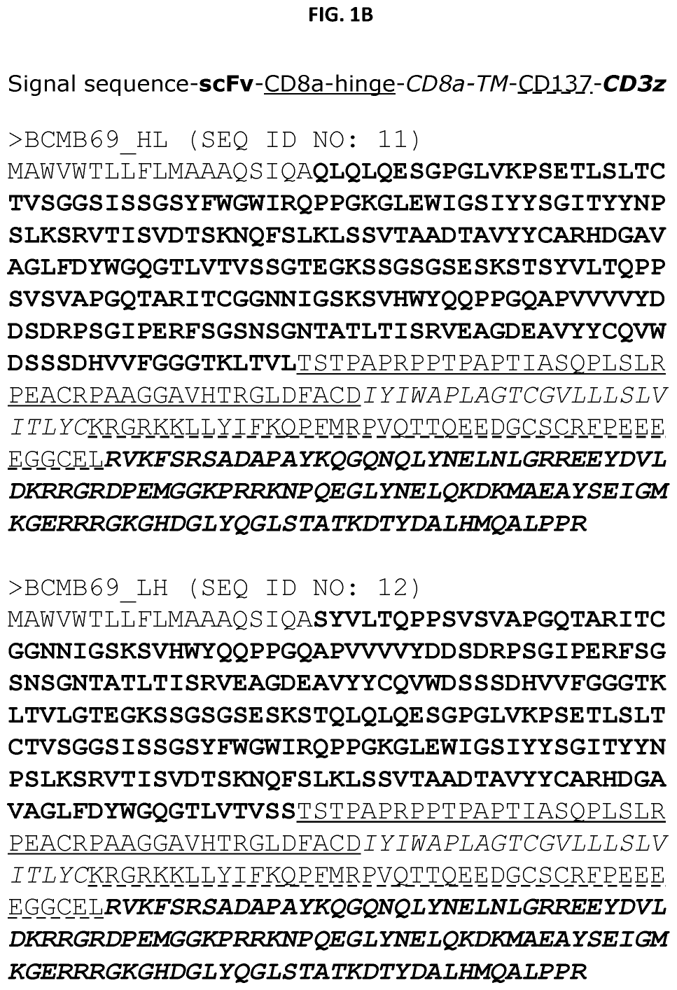

Second generation CAR-T of the present Example comprised a single-chain variable fragment (scFv) targeting a tumor associated antigen(s) (TAA) of interest, e.g., B-cell maturation antigen (BCMA), fused with a hinge and transmembrane sequence derived from human CD8A and intracellular domains e.g., 4-1BB and CD3C, as shown in FIG. 1A. scFvs for CAR-T constructs were specifically designed to include 5′ and 3′ overlap, corresponding to the EcoRI and SpeI restriction sites in a viral vector, e.g., a lentiviral vector. The designed DNA inserts were codon-optimized for Homo sapiens and subsequently synthesized. Cloning of constructs was performed using an In-Fusion® cloning method. All constructs were sequence-confirmed prior to transfection. Sequences of the BCMA CAR-T constructs used in the experiments presented herein are shown in FIG. 1B.

Below is a list of reagents useful in any of the CAR-T cell Assay methods disclosed herein.

### Reagents:

TransAct™
Naïve pan T cells
Costar® 24well Clear TC-treated Multiple Well Plates
Mouse anti-human CD4 antibody
Mouse anti-human CD8 antibody
Mouse anti-human CD62L antibody
Mouse anti-human CCR7 antibody
Mouse anti-human CD27 antibody
Anti-human CD45RO antibody
Anti-human CD45RA antibody
Anti-human CD279 (PD-1)
Anti-human CD233 (LAG-3)
Anti-human CD366 (Tim-3)
Anti-human TIGIT

### Example 2. Human T Cells Culture and Electroporation

Human Pan-T cells were isolated from peripheral blood monocyte cells (PBMCs) of healthy donors, and cultured in complete T cell media/RPMI media supplemented with 10% Fetal Calf Serum (FCS), 2 mM GlutaMax, 1 mM sodium pyruvate, 55 μM β-mercaptoethanol, and 100U penicillin/streptomycin.

Pan-T cells were expanded ex vivo using magnetic Dynabeads of anti-CD3/CD28 for about 12-14 days following manufacturer's protocol. The cells were then frozen at a density of 1×10⁶ cells/vial, and stored in liquid nitrogen.

### Example 3. T Cell Activation and Chimeric Antigen Receptor (CAR) Transduction

At day 0, naïve Pan-T cells from three donors were thawed and diluted in complete T cell media/RPMI media (e.g., see Example 2) to a density of 1×10⁶ T cells/ml. Prior to T cell activation, the thawed naïve T cells were immunophenotyped. The immunophenotype panel included, e.g., CD4⁺ and CD8⁺ T cell subsets. For T cell activation, 10 μl of TransAct™/ml was added to each well of a 24-well plate, with a density of 1×10⁶ T cells/well. The cells were incubated overnight at 37° C./5% C02. At day 1, the cells were transduced with B-cell maturation antigen (BCMA)-HL CAR lentiviral particles at a multiplicity of infection (MOI) equal to 5.

### Example 4. Phenotypic Characterization of BCMA-HL CAR Transduced T Cells

CAR-transduced T cells were expanded for 14 days using cytokine conditioning to enhance stem cell-like memory T cell (T_{SCM}) phenotype in CD4⁺ and CD8⁺ T cell subsets. Specifically, next-generation T_{SCM} like cells were generated using cytokines, e.g., either in the presence of IL-7 alone, or in the presence of IL-7 in combination with IL-15 (IL-7+IL-15) or with IL-15 and IL-21 (IL7+IL-15 and IL-21).

For phenotypic characterization, CAR T cells were assessed for memory and effector T cell markers using Fluorescence-Activated Cell Sorting (FACS) flow cytometry analysis at day 14 after transduction. For FACS staining, 100 μl cells/well from BCMA-HL CAR-transduced cells were washed twice with phosphate buffered saline (PBS), and then cell marker-specific antibodies labeled with fluorescent conjugates were used. Antibodies used in the present experiment were each diluted at 1:200, and included, e.g., CD4, CD8, FVD (live dead), CD27, CD62L, CD45RA, CD45RO, CCR7, CD69 and CAR+ (1 μg/ml), as well as an Alexa Fluor™ 647 (AF647) secondary antibody. All samples were assessed using a Fortessa cell sorter system.

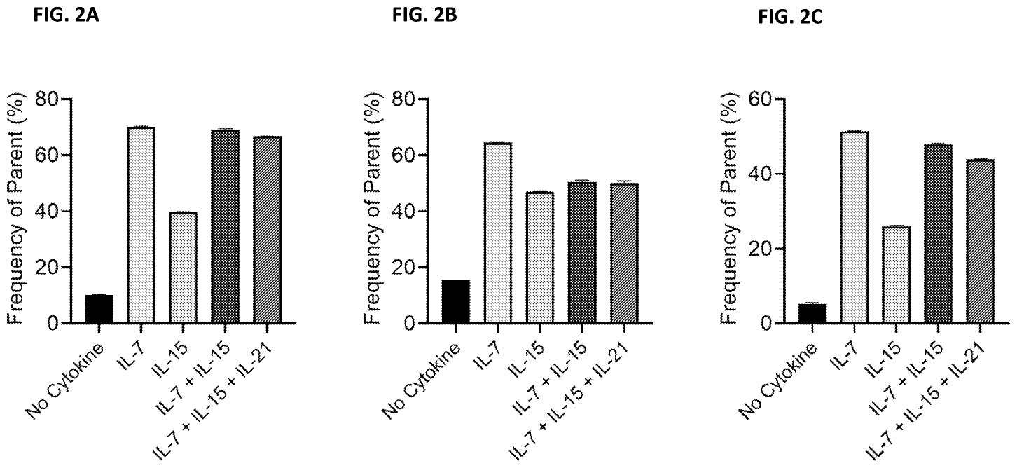

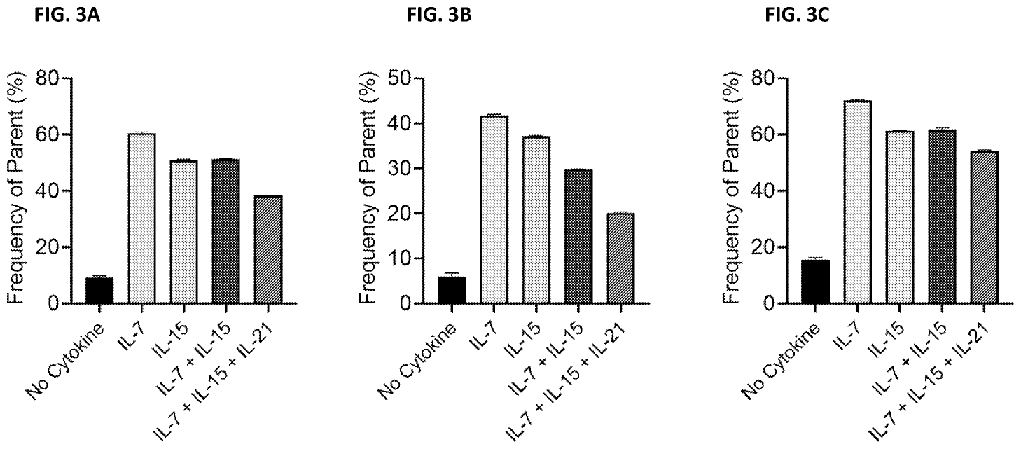

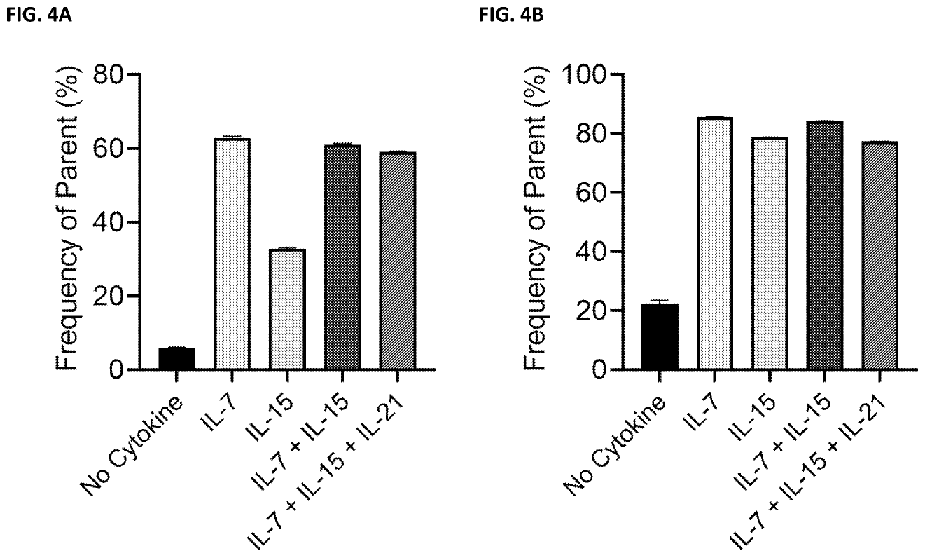

Representative cytokine enhancement of T_{SCM} cell phenotype in a CD4⁺ CAR-T cell subset and in a CD8⁺ CAR-T cell subset at day 14 after transduction are displayed in FIGS. 2A-2C and FIGS. 3A-3C, respectively. For phenotypic characterization of CD4⁺ CAR-T cells (FIGS. 2A-2C), pan-T cells were activated in the presence or absence of cytokines according to the above-described cytokine conditioning paradigm. For FACS analysis, the cells were gated on CD4⁺ CAR⁺ T cells and frequency of the expression level (frequency of parent, %) for CD62L (FIG. 2A), CCR7 (FIG. 2B), and CD27 (FIG. 2C) within the CD4⁺ BCMA-HL CAR transduced cells were determined. For phenotypic characterization of CD8⁺ CAR-T cells (FIGS. 3A-3C), pan-T cells were similarly activated in the presence or absence of cytokines according to the above-described cytokine conditioning paradigm. For FACS analysis, the cells were gated on CD8⁺ CAR⁺ T cells, and the frequency of the expression level (frequency of parent, %) for CD62L (FIG. 3A), CCR7 (FIG. 3B), and CD27 (FIG. 3C) within the CD8⁺ BCMA-HL CAR transduced cells were determined. As shown in FIGS. 4A-B, phenotypic characterization was also performed according to the same methods as those described above for quantification of the frequency of expression levels (frequency of parent, %) of CD45RO⁻/CD45RA⁺ in both CD4⁺ CAR-T cell (FIG. 4A) and CD8⁺ CAR-T cell (FIG. 4B) subsets. Cytokine conditioning enhanced CD45RO⁻/CD45RA⁺ T_{SCM} cell phenotype in both CD4⁺ and CD8⁺ CAR-T cell subsets.

Based on above results, a single cytokine (IL-7) effectively enriched T_{SCM} like cells in both CD4⁺ and CD8⁺ subsets. It was further observed that when IL-15 and IL-21 were added to IL-7 cytokine conditioning, T_{SCM} phenotype in both CD4⁺ and CD8⁺ T cells was enhanced. When IL-7+IL-15+IL-21 cytokine conditioned CAR T cells were co-cultured with tumor targets, IL-7+IL-15+IL-21 CAR T cells have enhanced cytokine production. While not wishing to be bound by theory, it is believed that the addition of IL-15 and IL-21 activates STAT5 and STAT3 signaling respectively which might be advantageous compared to single cytokine conditioning.

The above-described approach for generating cytokine-conditioned CAR-T cells with enhanced effector function and decreased exhaustion markers demonstrated proof-of-concept of generating both CD4⁺ and CD8⁺ CAR-T_{SCM} cells to enhance anti-tumor immunity.

### REFERENCES

1. June C H, O'Connor R S, Kawalekar O U, Ghassemi S, Milone M C. CAR T cell immunotherapy for human cancer. Science (New York, N Y) 2018; 359(6382):1361-5 doi 10.1126/science.aar6711.
2. Mirzaei H R, Rodriguez A, Shepphird J, Brown C E, Badie B. Chimeric Antigen Receptors T Cell Therapy in Solid Tumor: Challenges and Clinical Applications. Front Immunol 2017; 8:1850 doi 10.3389/fimmu.2017.01850.
3. Anderson J K, Mehta A. A review of chimeric antigen receptor T-cells in lymphoma. Expert Rev Hematol 2019; 12(7):551-61 doi 10.1080/17474086.2019.1629901.
4. Minutolo N G, Hollander E E, Powell D J, Jr. The Emergence of Universal Immune Receptor T Cell Therapy for Cancer. Front Oncol 2019; 9:176 doi 10.3389/fonc.2019.00176.
5. Kloss C C, Condomines M, Cartellieri M, Bachmann M, Sadelain M. Combinatorial antigen recognition with balanced signaling promotes selective tumor eradication by engineered T cells. Nat Biotechnol 2013; 31(1):71-5 doi 10.1038/nbt.2459.
6. Brudno I N, Kochenderfer J N. Toxicities of chimeric antigen receptor T cells: recognition and management. Blood 2016; 127(26):3321-30 doi 10.1182/blood-2016-04-703751.
7. Porter D L, Hwang W T, Frey N V, Lacey S F, Shaw P A, Loren A W, et al. Chimeric antigen receptor T cells persist and induce sustained remissions in relapsed refractory chronic lymphocytic leukemia. Sci Transl Med 2015; 7(303):303ra139 doi 10.1126/scitranslmed.aac5415.
8. Eshhar Z, Waks T, Gross G, Schindler D G. Specific activation and targeting of cytotoxic lymphocytes through chimeric single chains consisting of antibody-binding domains and the gamma or zeta subunits of the immunoglobulin and T-cell receptors. Proceedings of the National Academy of Sciences of the United States of America 1993; 90(2):720-4 doi 10.1073/pnas.90.2.720.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A method of enriching stem-cell like memory T (T_{SCM}) cells in a population of T cells, comprising the following step(s):
a) contacting the population of T cells with an effective amount of one or more cytokines comprising Interleukin 7 (IL-7) for a period of time sufficient to enrich T_{SCM} cells; and
b) optionally expanding said T_{SCM} cells.

2. The method of claim 1, wherein the one or more cytokines further comprise IL-15.

3. The method of claim 1, wherein the one or more cytokines further comprise IL-21.

4. The method of claim 1, wherein the one or more cytokines further comprise IL-15 and IL-21.

5. The method of claim 1, wherein each of the one or more cytokines is contacted with the population of T cells at a concentration of about 1 to about 15 ng/ml.

6. The method of claim 1, wherein each of the one or more cytokines is contacted with the population of T cells at a concentration of about 5 to 12 ng/ml.

7. The method of claim 1, wherein each of the one or more cytokines is contacted with the population of T cells at a concentration of about 10 ng/ml.

8. The method of claim 1, wherein the one or more cytokines do not comprise IL-2.

9. The method of claim 1, wherein the population of T cells comprises Pan T cells, naïve CD4⁺ T cells, naïve CD8⁺ T cells, or naïve CD4⁺ and naïve CD8⁺ T cells, or any combination thereof.

10. The method of claim 9, wherein the method further comprises isolating Pan T cells, naïve CD4⁺ cells, naïve CD8⁺ T cells, or naïve CD4⁺ and naïve CD8⁺ cells from peripheral blood monocyte cells (PBMC) prior to step (a).

11. The method of claim 1, wherein the population of T cells does not comprise inhibitory, regulatory T cells.

12. The method of claim 1, wherein said one or more cytokines are present during the expansion step (b).

13. The method of claim 1, wherein the method further comprises:
genetically modifying the T cells to express a chimeric antigen receptor (CAR) or engineered T cell receptor (TCR).

14. The method of claim 13, wherein said genetic modification is conducted by introducing into the cells a polynucleotide encoding said CAR or engineered TCR.

15. The method of claim 14, wherein the polynucleotide encoding said CAR or engineered TCR is introduced via viral transduction, electroporation, direct injection, magnetofection, ultrasound, a ballistic or hydrodynamic method, or a combination thereof.

16. The method of claim 13, wherein the CAR or engineered TCR specifically binds a tumor antigen, an infectious antigen or an autoimmune antigen.

17. The method of claim 16, wherein the tumor antigen is selected from BCMA, GPRC5D, CD79, KLK2, CD19, CD30, CD33, CD123, hK2, FLT3, CD20, CD22, KRASG12D, p53, BRAC1, and PSMA.

18. The method of claim 1, wherein said genetic modification is conducted prior to the expansion step (b).

19. The method of claim 18, wherein said one or more cytokines are present during the genetic modification step.

20. The method of claim 1, wherein the contacting step (a) is performed for 10-20 days.

21. The method of claim 1, wherein the contacting step (a) is performed for about 14 days.

22. The method of claim 1, wherein the contacting step (a) and the expansion step (b) are performed for a total of 10-20 days-.

23. The method of claim 1, wherein the contacting step (a) and the expansion step (b) are performed for a total of 14 days.

24. The method of claim 1, wherein the contacting step (a) is performed at a temperature of about 37° C.

25. The method of claim 1, wherein the method further comprises:
activating the population of T cells at the beginning of the contacting step (a).

26. The method of claim 1, wherein the activating step is performed with an anti-CD3 agent and/or an anti-CD28 agent for about 24 hours.

27. The method of claim 25, wherein the method further comprises:
priming the population of T cells prior to the activating step (a).

28. The method of claim 1, wherein the method further comprises:
determining the percentage of T_{SCM} cells in the population of T cells after the expansion step (b).

29. The method of claim 28, wherein the percentage of T_{SCM} cells in the population of T cells is at least about 40%, 50%, 60%, or 70% after the expansion step (b).

30. The method of claim 29, wherein the percentage of T_{SCM} cells in the population of T cells is about 60%-70% after the expansion step (b).

31. A method of generating genetically modified stem-cell like memory T (T_{SCM}) cells, comprising the following steps:
a) obtaining a population of isolated Pan T cells, naïve CD4⁺ T cells, naïve CD8⁺ T cells, or naïve CD4⁺ and naïve CD8⁺ T cells, or any combination thereof;
b) activating said population of T cells;
c) genetically modifying the cells present after step (b) to express a chimeric antigen receptor (CAR) or engineered T cell receptor (TCR);
d) expanding said genetically modified cells;
wherein steps b), c) and d) are performed in the presence of one or more cytokines comprising Interleukin-7 (IL-7).

32. The method of claim 31, wherein the one or more cytokines further comprise IL-15.

33. The method of claim 31, wherein the one or more cytokines further comprise IL-21.

34. The method of claim 31, wherein the one or more cytokines further comprise IL-15 and IL-21.

35. The method of claim 31, wherein each of the one or more cytokines is contacted with the population of T cells at a concentration of about 1 to about 15 ng/ml.

36. The method of claim 31, wherein each of the one or more cytokines is contacted with the population of T cells at a concentration of about 5 to 12 ng/ml.

37. The method of claim 31, wherein each of the one or more cytokines is contacted with the population of T cells at a concentration of about 10 ng/ml.

38. The method of claim 31, wherein the one or more cytokines do not comprise IL-2.

39. The method of claim 31, wherein the Pan T cells, naïve CD4⁺ T cells, naïve CD8⁺ T cells, or naïve CD4⁺ and naïve CD8⁺ T cells, or any combination thereof, are isolated from peripheral blood monocyte cells (PBMC).

40. The method of claim 39, wherein the T cells do not comprise inhibitory, regulatory T cells.

41. The method of claim 31, wherein said genetic modification is conducted by introducing into the cells a polynucleotide encoding said CAR or engineered TCR.

42. The method of claim 41, wherein the polynucleotide encoding said CAR or engineered TCR is introduced via viral transduction, electroporation, direct injection, magnetofection, ultrasound, a ballistic or hydrodynamic method, or a combination thereof.

43. The method of claim 31, wherein the CAR or engineered TCR specifically binds a tumor antigen, an infectious antigen or an autoimmune antigen.

44. The method of claim 43, wherein the tumor antigen is selected from BCMA, GPRC5D, CD79, KLK2, CD19, CD30, CD33, CD123, hK2, FLT3, CD20, CD22, KRASG12D, p53, BRAC1 and PSMA.

45. The method of claim 31, wherein the expansion step (d) is performed for 10-20 days.

46. The method of claim 31, wherein the expansion step (d) is performed for about 14 days.

47. The method of claim 31, wherein the steps (b), (c) and (d) are performed for a total of 10-20 days.

48. The method of claim 31, wherein the steps (b), (c) and (d) are performed for a total of 14 days.

49. The method of claim 31, wherein the steps (b), (c) and (d) are performed at a temperature of about 37° C.

50. The method of claim 31, wherein the activating step is performed with an anti-CD3 agent and/or an anti-CD28 agent for about 24 hours.

51. The method of claim 31, wherein the method further comprises:
priming the population of T cells prior to the activating step (b).

52. The method of claim 31, wherein the method further comprises:
determining the percentage of T_{SCM} cells in the population of T cells after the expansion step (d).

53. The method of claim 52, wherein the percentage of T_{SCM} cells in the population of T cells is at least about 40%, 50%, 60%, or 70% after the expansion step (d).

54. The method of claim 53, wherein the percentage of T_{SCM} cells in the population of T cells is about 60%-70% after the expansion step (d).

55. A population of T cells comprising enriched stem-cell like memory T (T_{SCM}) cells, prepared by a method according to claim 1.

56. A pharmaceutical composition comprising the population of T cells of claim 55, and a pharmaceutically acceptable carrier or excipient.

57. A method of treating a disease or disorder in a subject in need thereof, comprising administering to the subject a therapeutically effective amount of the population of T cells of claim 55.

58. The method of claim 57, wherein the population of T cells are allogeneic to the subject.

59. The method of claim 57, wherein the population of T cells are autologous to the subject.

60. The method of claim 57, wherein the disease or disorder is a cancer, an infectious disease, or an autoimmune disease.

61. The method of claim 60, wherein the cancer is a hematologic malignancy.

62. The method of claim 60, wherein the cancer is a solid tumor.

63. The method of claim 60, wherein the cancer is squamous cell cancer, adenosquamous cell carcinoma, lung cancer, cancer of the peritoneum, hepatocellular cancer, gastric or stomach cancer, cervical cancer, ovarian cancer, liver cancer, bladder cancer, cancer of the urinary tract, hepatoma, breast cancer, colon cancer, colorectal cancer, endometrial cancer, salivary gland carcinoma, kidney or renal cancer, prostate cancer, vulval cancer, thyroid cancer, hepatic carcinoma, anal carcinoma, penile carcinoma, skin cancer, multiple myeloma and acute lymphocytic leukemia (ALL), acute myelocytic leukemia (AML), chronic myelocytic leukemia (CML), and chronic lymphocytic leukemia (CLL), lymphoma such as Hodgkin lymphoma (HL) and non-Hodgkin lymphoma (NHL), follicular lymphoma, chronic lymphocytic leukemia/small lymphocytic lymphoma (CLL/SLL), mantle cell lymphoma (MCL), marginal zone B-cell lymphomas, primary mediastinal B-cell lymphoma, Burkitt lymphoma, lymphoplasmacytic lymphoma, immunoblastic large cell lymphoma, hairy cell leukemia (HCL), precursor B-lymphoblastic lymphoma and primary central nervous system (CNS) lymphoma, T-cell NHL such as precursor T-lymphoblastic lymphoma/leukemia, peripheral T-cell lymphoma (PTCL), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma, brain, as well as head and neck cancer, biliary cancer, bronchus cancer, chordoma, choriocarcinoma, epithelial carcinoma, endothelial sarcoma, esophageal cancer, Ewing sarcoma, heavy chain disease, hematopoietic cancer, immunocytic amyloidosis, monoclonal gammopathy of undetermined significance, myelodysplastic syndromes, myeloproliferative disorder, agnogenic myeloid metaplasia (AMM) or myelofibrosis (MF), chronic idiopathic myelofibrosis, myeloproliferative neoplasms, polycythemia vera, rectum adenocarcinoma, essential thrombocytosis, chronic neutrophilic leukemia, hypereosinophilic syndrome, or soft tissue sarcoma, or a combination or metastases thereof.

64. The method of claim 60, wherein the cancer is a BCMA-expressing cancer.

65. The method of claim 64, wherein the BCMA-expressing cancer is acute myeloid leukemia (AML), multiple myeloma (MM), or smoldering multiple myeloma (SMM).

66. A system for enriching stem-cell like memory T (T_{SCM}) cells in a population of T cells, comprising the following elements:
a) a means for contacting the population of T cells with an effective amount of one or more cytokines comprising Interleukin 7 (IL-7) for a period of time sufficient to enrich T_{SCM} cells; and
b) optionally a means for expanding said T_{SCM} cells.

67. The system of claim 66, wherein the population of T cells comprises Pan T cells, naïve CD4⁺ T cells, naïve CD8⁺ T cells, or naïve CD4⁺ and naïve CD8⁺ T cells, or any combination thereof.

68. The system of claim 67, wherein the system further comprises means for isolating Pan T cells, naïve CD4⁺ cells, naïve CD8⁺ T cells, or naïve CD4⁺ and naïve CD8⁺ cells, or any combination thereof, from peripheral blood monocyte cells (PBMC).

69. The system of claim 66, where the system further comprises means for genetically modifying the T cells to express a chimeric antigen receptor (CAR) or engineered T cell receptor (TCR).

70. The system of claim 66, wherein the system further comprises means for activating the population of T cells at the beginning of the contacting said population of T cells with the one or more cytokines comprising Interleukin 7 (IL-7).

71. The system of claim 70, wherein the system further comprises means for priming the population of T cells prior to the activating.

72. The system of claim 66, wherein the system further comprises means for
determining the percentage of T_{SCM} cells in the population of T cells after the expansion.

73. A system for generating genetically modified stem-cell like memory T (T_{SCM}) cells, comprising the following elements:
a) means for obtaining a population of isolated Pan T cells, naïve CD4⁺ T cells, naïve CD8⁺ T cells, or naïve CD4⁺ and naïve CD8⁺ T cells, or any combination thereof;
b) means for activating said population of T cells;
c) means for genetically modifying the cells after activating to express a chimeric antigen receptor (CAR) or engineered T cell receptor (TCR);
d) means for expanding said genetically modified cells;
wherein obtaining a population of isolated Pan T cells, naïve CD4⁺ T cells, naïve CD8⁺ T cells, or naïve CD4⁺ and naïve CD8⁺ T cells, or any combination thereof, activating said population of T cells and genetically modifying said population of T cells is performed in the presence of one or more cytokines comprising Interleukin-7 (IL-7).

74. The system of claim 66, wherein the one or more cytokines further comprise IL-15 and/or IL-21.

75. A composition for enriching stem-cell like memory T (T_{SCM}) cells in a population of T cells, comprising:
a. one or more cytokines comprising Interleukin 7 (IL-7) for enriching T_{SCM} cells,
b. a population of T cells, which is a means for enriching T_{SCM} cells and
c. optionally a means for expanding said T_{SCM} cells.

76. A composition for enriching stem-cell like memory T (T_{SCM}) cells in a population of T cells, comprising:
a. a population of T cells, and
b. an effective amount of one or more cytokines comprising Interleukin 7 (IL-7), and
a means for:
(i) contacting the population of T cells with an effective amount of one or more cytokines comprising IL-7 thereby enriching T_{SCM} cells,
(ii) activating said enriched T_{SCM} cells, and
(iii) optionally a means for expanding said T_{SCM} cells.

77. A composition for generating genetically modified stem-cell like memory T (T_{SCM}) cells, comprising:
a. a population of T cells, and
b. an effective amount of one or more cytokines comprising Interleukin 7 (IL-7), and
a means for:
(i) contacting the population of T cells with an effective amount of one or more cytokines comprising IL-7 thereby enriching T_{SCM} cells,
(ii) activating said enriched T_{SCM} cells,
(iii) genetically modifying said enriched T_{SCM} cells to express a chimeric antigen receptor (CAR) or engineered T cell receptor (TCR), and
(iv) optionally a means for expanding said T_{SCM} cells.

78. The composition of claim 75, wherein the T_{SCM} cells are enriched by placing the population of T cells in contact with an effective amount of the one or more cytokines comprising IL-7 for a period of time sufficient to enrich T_{SCM} cells.


SEQUENCE LISTING (12 sequences)

SEQ ID NO: 1 - protein, 19 aa, Artificial Sequence
  source: /note=“Description of Artificial Sequence Synthetic peptide”
MAWVWTLLFLMAAAQSIQA

SEQ ID NO: 2 - protein, 245 aa, Artificial Sequence
  source: /note=“Description of Artificial Sequence Synthetic polypeptide”
QLQLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSSGTEGKSSGSGSESKSTSYVLTQPPSVSVAPGQTARITCGGNNIGSKSVHWYQQPPGQAPVVVVYDDSDRPSGIPERFSGSNSGNTATLTISRVEAGDEAVYYCQVWDSSSDHVVFGGGTKLTVL

SEQ ID NO: 3 - protein, 245 aa, Artificial Sequence
  source: /note=“Description of Artificial Sequence Synthetic polypeptide”
SYVLTQPPSVSVAPGQTARITCGGNNIGSKSVHWYQQPPGQAPVVVVYDDSDRPSGIPERFSGSNSGNTATLTISRVEAGDEAVYYCQVWDSSSDHVVFGGGTKLTVLGTEGKSSGSGSESKSTQLQLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSS

SEQ ID NO: 4 - protein, 16 aa, Artificial Sequence
  source: /note=“Description of Artificial Sequence Synthetic peptide”
GTEGKSSGSGSESKST

SEQ ID NO: 5 - protein, 45 aa, Artificial Sequence
  source: /note=“Description of Artificial Sequence Synthetic polypeptide”
TSTPAPRPPTPAPTIASQPLSLRPEACRPAAGGAVHTRGLDFACD

SEQ ID NO: 6 - protein, 24 aa, Artificial Sequence
  source: /note=“Description of Artificial Sequence Synthetic peptide”
IYIWAPLAGTCGVLLLSLVITLYC

SEQ ID NO: 7 - protein, 42 aa, Artificial Sequence
  source: /note=“Description of Artificial Sequence Synthetic polypeptide”
KRGRKKLLYIFKQPFMRPVQTTQEEDGCSCRFPEEEEGGCEL

SEQ ID NO: 8 - protein, 112 aa, Artificial Sequence
  source: /note=“Description of Artificial Sequence Synthetic polypeptide”
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 309 aa, Artificial Sequence
  source: /note=“Description of Artificial Sequence Synthetic polypeptide”
MAWVWTLLFLMAAAQSIQAQLQLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSSGTEGKSSGSGSESKSTSYVLTQPPSVSVAPGQTARITCGGNNIGSKSVHWYQQPPGQAPVVVVYDDSDRPSGIPERFSGSNSGNTATLTISRVEAGDEAVYYCQVWDSSSDHVVFGGGTKLTVLTSTPAPRPPTPAPTIASQPLSLRPEACRPAAGGAVHTRGLDFACD

SEQ ID NO: 10 - protein, 154 aa, Artificial Sequence
  source: /note=“Description of Artificial Sequence Synthetic polypeptide”
KRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 487 aa, Artificial Sequence
  source: /note=“Description of Artificial Sequence Synthetic polypeptide”
MAWVWTLLFLMAAAQSIQAQLQLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSSGTEGKSSGSGSESKSTSYVLTQPPSVSVAPGQTARITCGGNNIGSKSVHWYQQPPGQAPVVVVYDDSDRPSGIPERFSGSNSGNTATLTISRVEAGDEAVYYCQVWDSSSDHVVFGGGTKLTVLTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 487 aa, Artificial Sequence
  source: /note=“Description of Artificial Sequence Synthetic polypeptide”
MAWVWTLLFLMAAAQSIQASYVLTQPPSVSVAPGQTARITCGGNNIGSKSVHWYQQPPGQAPVVVVYDDSDRPSGIPERFSGSNSGNTATLTISRVEAGDEAVYYCQVWDSSSDHVVFGGGTKLTVLGTEGKSSGSGSESKSTQLQLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSSTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR